(19) United States
(12) Patent Application Publication
(10) Pub. No.: US 2025/0312267 A1
(43) Pub. Date: Oct. 9, 2025

(54) COSMETIC COMPOSITIONS AND METHODS OF USING SAME

(71) Applicant: MARY KAY INC., Addison, TX (US)

(72) Inventors: Ricky Mendoza, Addison, TX (US); Kathlene Cummings, Addison, TX (US); Lorie Abdul, Addison, TX (US)

(73) Assignee: MARY KAY INC., Addison, TX (US)

(21) Appl. No.: 19/170,723

(22) Filed: Apr. 4, 2025

Related U.S. Application Data
(60) Provisional application No. 63/575,478, filed on Apr. 5, 2024.

(51) Int. Cl. A61K 8/894; A61K 8/81; A61Q 1/02

(52) U.S. Cl. CPC A61K 8/894; A61K 8/8152; A61Q 1/02; A61K 2800/43; A61K 2800/594; A61K 2800/61; A61K 2800/651

(57) ABSTRACT

The present invention relates generally to methods and compositions useful for cosmetic purposes and include compositions comprising a dimethicone, at least one film-forming agent, and at least one surface-treated pigment, wherein the film-forming agent comprises a dimethicone acrylate copolymer, a trimethicone and/or isobutylmethacrylate/bis-hydroxypropyl dimethicone copolymer.

### CROSS-REFERENCE TO RELATED APPLICATIONS

This application claims the benefit of priority to U.S. Provisional Patent Application Ser. No. 63/575,478, filed Apr. 5, 2024, hereby incorporated by reference in its entirety.

### BACKGROUND OF THE INVENTION

### A. Field of the Invention

The present invention relates generally to cosmetic compositions that can be used to achieve fast drying spreadable foundations with good coverage and color fidelity. The composition can include a dimethicone, at least one film-forming agent, and at least one surface-treated pigment.

### B. Description of Related Art

Cosmetic compositions are commonly used to camouflage or even up dyschromia (such as freckles, age spots, lentigines, melasma, liver spots, chloasma, etc.). Cosmetic compositions also are used to camouflage or even up skin imperfections such as pores, wrinkles, spots and/or fine lines and/or acne scars. In this regard, many solid or fluid, anhydrous or non-anhydrous formulations have been developed to date. Coverage is a critical and desirable property to provide a correcting effect for skin imperfections.

Achieving highly spreadable foundations with good coverage and color fidelity is an inherent challenge with formulating pigment dispersions to deliver chromatophores.

### SUMMARY OF THE INVENTION

The present invention overcomes deficiencies in the art by providing stable foundation compositions that can also effectively maintain good coverage and color fidelity. Certain aspects herein use a low molecular weight dimethicone combined with a trimethicone acrylate copolymer, which can deliver color dispersion across the skin surface and can provide and maintain high color fidelity.

In some instances, there is disclosed a topical skin composition. In some instances, the compositions is a makeup composition. In some instances, the composition above includes any one of, any combination of, or all of a dimethicone, at least one film-forming agent, and at least one surface-treated pigment. The amounts of the ingredients within the composition can vary (e.g., amounts can be as low as 0.000001% to as high as 80% w/w or any range therein).

In some instances, the composition includes a dimethicone, at least one surface-treated pigment, and a dimethicone acrylate copolymer and/or a trimethicone and/or isobutylmethacrylate/bis-hydroxypropyl dimethicone copolymer. In some instances, the composition includes a dimethicone, at least one surface-treated pigment, and a dimethicone acrylate copolymer. In some instances, the composition includes a dimethicone, at least one surface-treated pigment, and a trimethicone. In some instances, the composition includes a dimethicone, at least one surface-treated pigment, and a isobutylmethacrylate/bis-hydroxypropyl dimethicone copolymer.

In some instances, the dimethicone comprises a low-molecular weight dimethicone. In some instances, the dimethicone has a molecular weight below 35,000 g/mol. In some instances, the dimethicone has a viscosity between 0.6-10 centistokes. In some instances, the film-forming agent comprises a dimethicone acrylate copolymer and a trimethicone. In some instances, the trimethicone comprises a methyl trimethicone. In some instances, the film-forming agent comprises an isobutylmethacrylate/bis-hydroxypropyl dimethicone copolymer. In some instances, the film-forming agent comprises a dimethicone acrylate copolymer dissolved in methyl trimethicone. In some instances, the film-forming agent comprises a isobutylmethacrylate/bis-hydroxypropyl dimethicone copolymer dissolved in isoparaffin.

In some instances, the surface-treated pigments comprise a metal oxide. In some instances, the surface-treated pigments are in a dimethicone dispersion.

The composition may further comprise one or more ingredients described herein. For example, the composition may comprise one or more additional ingredients selected from one or more conditioning agents, moisturizing agents, pH adjusters, structuring agents, inorganic salts, and preservatives. In some instances, the composition comprises a soft-focus agent. In some instances, the composition comprises a mattifying agent. In some instances, the composition comprises a luminous agent.

It is also specifically contemplated that one or more of the ingredients disclosed herein may be specifically excluded from a composition, in some instances. In some instances, the composition does not comprise decamethylcyclopentasiloxane. In some instances, the composition does not comprise a resin.

Methods of use for the compositions disclosed herein are also disclosed. In one aspect, the compositions disclosed are topical compositions. In some instances, the method comprises topically applying any one of the compositions disclosed herein to skin and/or the face and/or eye area in need thereof. In one aspect, any one of the compositions disclosed herein are topically applied and the composition is left on the application area, removed from the application area after a period of time, and/or removed directly after application. In some instances, applying the composition results in camouflaging of dyschromia and/or skin imperfections on the individual. In some instances, applying the composition results in a luminous effect on the skin of the individual. In some instances, applying the composition results in a mattifying effect on the skin of the individual.

In certain methods, the skin of the individual is oily. In certain methods, the topical skin composition absorbs sebum on the skin of the individual.

In particular aspects, the compositions of the present invention are formulated as a topical skin composition. The composition can have a dermatologically acceptable vehicle or carrier for the compounds, compositions and extracts. The composition can further include a moisturizing agent or a humectant, a surfactant, a silicone containing compounds, a UV agent, an oil, and/or other ingredients identified in this specification or those known in the art. The composition can be a lotion, cream, body butter, mask, scrub, wash, gel, serum, emulsion (e.g., oil-in-water, water-in-oil, silicone-in-water, water-in-silicone, water-in-oil-in-water, oil-in-water-in-oil, oil-in-water-in-silicone, etc.), solutions (e.g., aqueous or hydro-alcoholic solutions), anhydrous bases (e.g., lipstick or a powder), ointments, milk, paste, aerosol, solid forms, eye jellies, etc. The composition can be in powdered form (e.g., dried, lyophilized, particulate, etc.). The composition can be formulated for topical skin application at least 1, 2, 3, 4, 5, 6, 7, or more times a day during use. In some aspects of the present invention, compositions can be storage stable or color stable, or both. It is also contemplated that the viscosity of the composition can be selected to achieve a desired result, e.g., depending on the type of composition desired, the viscosity of such composition can be from about 1 cps to well over 1 million eps or any range or integer derivable therein (e.g., 2 cps, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 10000000, cps, etc., as measured on a Brookfield Viscometer using a TC spindle at 2.5 rpm at 25° C.).

The compositions of the present invention can also be modified to have a desired oxygen radical absorbance capacity (ORAC) value. In certain non-limiting aspects, the compositions of the present invention or the component or extracts thereof identified throughout this specification can be modified to have an ORAC value per mg of at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 70, 80, 90, 95, 100, 200, 300, 400, 500, 600, 700, 800, 900, 1000, 2000, 3000, 4000, 5000, 6000, 7000, 8000, 9000, 10000, 15000, 20000, 30000, 50000, 100000 or more or any range derivable therein.

The compositions, in non-limiting aspects, can have a pH of about 6 to about 9. In some aspects, the pH can be 1, 2, 3, 4, 5, 6, 7, 8, 9, 10, 11, 12, 13, or 14. The compositions can include a triglyceride. Non-limiting examples include small, medium, and large chain triglycerides. In certain aspects, the triglyceride is a medium chain triglyceride (e.g., caprylic capric triglyceride). The compositions can also include preservatives. Non-limiting examples of preservatives include phenoxyethanol, methylparaben, propylparaben, or any mixture of thereof. In some embodiments, the composition is paraben-free.

Compositions of the present invention can have UVA and UVB absorption properties. The compositions can have an sun protection factor (SPF) of 2, 3, 4, 5, 6, 7, 8, 9, 10, 11, 12, 13, 14, 15, 20, 25, 30, 35, 40, 45, 50, 55, 60, or more, or any integer or derivative therein. The compositions can be sunscreen lotions, sprays, or creams.

The compositions of the present invention can also include any one of, any combination of, or all of the following additional ingredients: water, a conditioning agent, a chelating agent, a moisturizing agent, a pH adjuster, inorganic salts, a preservative, a thickening agent, a silicone containing compound, an essential oil, a structuring agent, a vitamin, a pharmaceutical ingredient, or an antioxidant, or any combination of such ingredients or mixtures of such ingredients. In certain aspects, the composition can include at least two, three, four, five, six, seven, eight, nine, ten, or more, or all of these additional ingredients identified in the previous sentence. Non-limiting examples of these additional ingredients are identified throughout this specification and are incorporated into this section by reference. The amounts of such ingredients can range from 0.0001% to 99.9% by weight or volume of the composition, or any integer or range in between as disclosed in other sections of this specification, which are incorporated into this paragraph by reference.

Kits that include the compositions of the present invention are also contemplated. In certain embodiments, the composition is comprised in a container. The container can be a bottle, dispenser, or package. The container can dispense a pre-determined amount of the composition. In certain aspects, the compositions is dispensed in a spray, mist, dollop, or liquid. The container can include indicia on its surface. The indicia can be a word, an abbreviation, a picture, or a symbol.

It is also contemplated that the compositions disclosed throughout this specification can be used as a leave-on or rinse-off composition. By way of example, a leave-on composition can be one that is topically applied to skin and remains on the skin for a period of time (e.g., at least 5, 6, 7, 8, 9, 10, 20, or 30 minutes, or at least 1, 2, 3, 4, 5, 6, 7, 8, 9, 10, 11, 12, 13, 14, 15, 16, 17, 18, 19, 20, 21, 22, 23 or 24 hours, or overnight or throughout the day). Alternatively, a rinse-off composition can be a product that is intended to be applied to the skin and then removed or rinsed from the skin (e.g., with water) within a period of time such as less than 5, 4, 3, 2, or 1 minute. An example of a rinse off composition can be a skin cleanser, shampoo, conditioner, or soap. An example of a leave-on composition can be a skin moisturizer, sunscreen, mask, overnight cream, or a day cream.

It is contemplated that any embodiment discussed in this specification can be implemented with respect to any method or composition of the invention, and vice versa. Furthermore, compositions of the invention can be used to achieve methods of the invention.

In the context of the present invention, at least the following 20 aspects are described: Aspect 1 includes a topical skin composition comprising a dimethicone, at least one film-forming agent, and at least one surface-treated pigment, wherein the film-forming agent comprises a dimethicone acrylate copolymer, a trimethicone, and/or isobutylmethacrylate/bis-hydroxypropyl dimethicone copolymer. Aspect 2 depends upon Aspect 1, wherein the dimethicone comprises a low-molecular weight dimethicone. Aspect 3 depends upon Aspect 1, wherein the film-forming agent comprises a dimethicone acrylate copolymer and a trimethicone. Aspect 4 depends upon Aspect 3, wherein the trimethicone comprises methyl trimethicone. Aspect 5 depends upon Aspect 1, wherein the film-forming agent comprises an isobutylmethacrylate/bis-hydroxypropyl dimethicone copolymer. Aspect 6 depends upon Aspect 1, wherein the film-forming agent comprises a dimethicone acrylate copolymer dissolved in methyl trimethicone. Aspect 7 depends upon Aspect 1, wherein the film-forming agent comprises a isobutylmethacrylate/bis-hydroxypropyl dimethicone copolymer dissolved in isoparaffin. Aspect 8 depends upon Aspect 1, wherein the surface-treated pigments comprise a metal oxide. Aspect 9 depends upon Aspect 1, wherein the surface-treated pigments are in a dimethicone dispersion. Aspect 10 depends upon Aspect 1, wherein the dimethicone is between 0.65 to 10 centistokes. Aspect 11 depends upon Aspect 1, wherein the composition further comprises a soft-focus agent. Aspect 12 depends upon Aspect 1, wherein the composition further comprises a mattifying agent. Aspect 13 depends upon Aspect 1, wherein the composition is an emulsion. Aspect 14 depends upon Aspect 1, wherein the composition does not comprise decamethylcyclopentasiloxane. Aspect 15 depends upon Aspect 1, wherein the composition does not comprise a resin. Aspect 16 includes a method of treating skin, the method comprising applying the topical skin composition of claim 1 topically to skin of an individual. Aspect 17 depends upon Aspect 16, wherein the applying results in camouflaging of dyschromia and/or skin imperfections on the individual. Aspect 18 depends upon Aspect 16, wherein the applying results in a luminous effect on the skin of the individual. Aspect 19 depends upon Aspect 16, wherein the applying results in a mattifying effect on the skin of the individual. Aspect 20 depends upon Aspect 16, wherein the skin of the individual is oily. Aspect 21 depends upon Aspect 16, wherein the topical skin composition absorbs sebum on the skin of the individual.

In some embodiments, compositions of the present invention can be pharmaceutically or cosmetically elegant or can have pleasant tactile properties. “Pharmaceutically elegant,” “cosmetically elegant,” and/or “pleasant tactile properties” describes a composition that has particular tactile properties which feel pleasant on the skin (e.g., compositions that are not too watery or greasy, compositions that have a silky texture, compositions that are non-tacky or sticky, etc.). Pharmaceutically or cosmetically elegant can also relate to the creaminess or lubricity properties of the composition or to the moisture retaining properties of the composition.

Also contemplated is a product comprising a composition of the present invention. In non-limiting aspects, the product can be a cosmetic product. The cosmetic product can be those described in other sections of this specification or those known to a person of skill in the art. Non-limiting examples of products include a moisturizer, a cream, a lotion, a skin softener, a serum, a gel, a wash, a body butter, a scrub, a foundation, a night cream, a lipstick, a cleanser, a toner, a sunscreen, a mask, an anti-aging product, a deodorant, an antiperspirant, a perfume, a cologne, etc.

“Topical application” means to apply or spread a composition onto the surface of lips or keratinous tissue. “Topical skin composition” includes compositions suitable for topical application on skin, lips, and/or keratinous tissue. Such compositions are typically dermatologically-acceptable in that they do not have undue toxicity, incompatibility, instability, allergic response, and the like, when applied to lips, skin, and/or keratinous tissue. Topical skin care compositions of the present invention can have a selected viscosity to avoid significant dripping or pooling after application to lips, skin, and/or keratinous tissue.

“Keratinous tissue” includes keratin-containing layers disposed as the outermost protective covering of mammals and includes, but is not limited to, lips, skin, hair and nails.

The term “about” or “approximately” are defined as being close to as understood by one of ordinary skill in the art. In one non-limiting embodiment the terms are defined to be within 10%, preferably within 5%, more preferably within 1%, and most preferably within 0.5%.

The term “substantially” and its variations are defined as being largely but not necessarily wholly what is specified as understood by one of ordinary skill in the art, and in one non-limiting embodiment substantially refers to ranges within 10%, within 5%, within 1%, or within 0.5%.

The terms “inhibiting” or “reducing” or any variation of these terms includes any measurable decrease or complete inhibition to achieve a desired result. The terms “promote” or “increase” or any variation of these terms includes any measurable increase, such as a measurable increase of a protein or molecule (e.g., matrix proteins such as fibronectin, laminin, collagen, or elastin or molecules such as hyaluronic acid) to achieve a desired result.

The term “effective,” as that term is used in the specification and/or claims, means adequate to accomplish a desired, expected, or intended result.

The use of the word “a” or “an” when used in conjunction with the terms “comprising,” “including,” “having,” or “containing,” or any variations of these terms, in the claims and/or the specification may mean “one,” but it is also consistent with the meaning of “one or more,” “at least one,” and “one or more than one.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. With respect to the phrase “consisting essentially of,” a basic and novel property of the compositions and methods of the present invention is the ability to have suitable coverage and color fidelity.

Other objects, features and advantages of the present invention will become apparent from the following detailed description. It should be understood, however, that the detailed description and the examples, while indicating specific embodiments of the invention, are given by way of illustration only. Additionally, it is contemplated that changes and modifications within the spirit and scope of the invention will become apparent to those skilled in the art from this detailed description.

### DESCRIPTION OF ILLUSTRATIVE EMBODIMENTS

As noted above, several of the unique aspects of the present invention are the use of a low molecular weight dimethicone combined with a trimethicone acrylate copolymer helped to deliver highly spreadable foundation across the skin's surface. This allows for the benefits of providing and maintaining high color fidelity and coverage. Aspects herein overcome the challenge of formulating fast drying spreadable foundations with good coverage and color fidelity while delivering a chromatophore. Additional agents that were combined to provide further optical effects, depending on the type of foundation targeted, such as either luminous or matte. These additional agents can be for various purposes including texture enhancing, soft-focus, blurring, and adding luminescence or mattifying for respective foundation types.

The following subsections describe non-limiting aspects of the present invention in further detail.

Some compositions disclosed herein are designed to work as a topical skin composition. The composition relies on a unique combination of any one of, any combination of, or all of a dimethicone, at least one film-forming agent, and at least one surface-treated pigment. Non-limiting examples of such a composition are provided in Example 1, Tables 1 and 2.

Some compositions disclosed herein can be applied to the skin or hair and remain on the skin or hair for a period of time (e.g., at least 1, 2, 3, 4, 5, 10, 20, 30, or 60 minutes or more). After which, the composition, if needed, can be rinsed from the skin or peeled from the skin. Some compositions disclosed herein can be applied to the skin and immediately rinsed from the skin. Some compositions disclosed herein can be applied to the skin and absorbed at least in part by the skin.

These and other non-limiting aspects of the present invention are described in the following sections.

### A. Active Ingredients

Aspects of the present disclosure include a combination of one or more of active ingredients—including dimethicone, at least one film-forming agent, and/or at least one surface-treated pigment—that can be used to correct skin imperfections.

Dimethicone is a silicone polymer, and is also referred to as polydimethylsiloxane or PDMS. In some instances, this ingredient is commercially available. It has been determined that this ingredient can be used to alter viscosity, in some instances. The dimethicone can have a viscosity between approximately 0.6 to 10 centistokes. In certain instances, the dimethicone has a viscosity of at least, at most, or approximately 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, or 10.9 centistokes, or any range derivable therein.

Film-forming agents include a variety of compositions, such as polymers or acrylates, that can allow for a composition to have a continuous, spreadable layer applied to the skin. Film-forming agents of the present disclosure can include a dimethicone acrylate copolymer, a trimethicone, and/or an isobutylmethacrylate/bis-hydroxypropyl dimethicone copolymer. The trimethicone can be methyl trimethicone.

Surface-treated pigments include pigments coated with polymers or other materials to improve desired characteristics for compositions and formulations herein. The pigments can be metal oxides. The pigments can be treated with or comprised in a dispersion, such as a dimethicone dispersion.

Soft-focus agents can be defined as materials, such as powders with high diffuse reflectance, low specular reflectance and high diffuse transmittance otherwise known as soft-focus powders or blurring agents give the skin a smoother appearance, by reducing the difference in luminosity between the valley and the edges of wrinkles and imperfections. Examples of soft-focus agents include powders of natural or synthetic origin such as mica, synthetic fluorophlogopite, titanated mica, alumina, aluminum silicate, non-porous and/or porous silica, fumed silica, silica silylate, cellulose, starch, titanium dioxide, zinc oxide, kaolin and sericite, composite titanium dioxide/alumina/silica powders, such as those sold under the trade name COVERLEAF AR-80™ by Catalyst & Chemicals, Japan. Non-mineral powders with a soft-focus effect include polyamide with a mean size of 10 microns as well as and other acrylate copolymers polyethylene, silk powder, polyurethane and boron nitride.

Mattifying agents are used to reduce the excessively shiny appearance of oily skin types by absorbing sebum and the excess non-volatile emollients in the composition not absorbed by the skin. Examples of mattifying agents include some of the soft-focus powders mentioned above which include, but are not limited to, natural mica, synthetic fluorophlogopite, alumina, aluminum silicate, porous and/or non-porous silica, fumed silica, silica silylate, cellulose, starch, zinc oxide, kaolin, sericite, and composites of these materials. Materials known to be porous generally have a higher potential to absorb oil. Mattifying powders may be coated or uncoated. Adding a coating to a powder is known to reduce the oil absorptivity. Care must be taken to select the appropriate grade of powder for the application. These agents will be employed for consumers with oily skin to give a natural matte appearance.

Luminescent agents are intended to allow the formula to reflect light mainly in the specular direction, possibly in uniformity over the entire area or face and that this light is not too intense like the gloss of a shiny oil or highly reflective in iridescence. Examples of natural looking luminous agents include silicones, high refractive emollients and/or ingredients such as high gloss film formers. Any powder with high reflectance, high specular reflectance and high transmittance otherwise known as luminous agents give the skin a glowing appearance by increasing the difference in luminosity with the skin's topographic areas such as pores and wrinkles. Examples include bismuth oxychloride, titanated mica, alumina, aluminum silicate, silica silylate, titanium dioxide, and sericite, natural or synthetic micas (fluorphlogopite), which could entail the use of superposed layer to multilayers of metal oxides and/or of organic colorants. Non-mineral powders with a high-focus effect include polyamides with a mean size of 10 microns as well as and other acrylate copolymers polyethylene, silk powder, polyurethane and boron nitride.

This combination of ingredients can be used in different product forms to treat various skin conditions. By way of non-limiting examples, the combination of ingredients can be formulated in an emulsion (e.g., oil in water, water in oil), a gel, a serum, a gel emulsion, a gel serum, a lotion, a mask, a scrub, a wash, a cream, or a body butter.

The extracts described herein can be extracts made through extraction methods known in the art and combinations thereof. Non-limiting examples of extraction methods include the use of liquid-liquid extraction, solid phase extraction, aqueous extraction, ethyl acetate, alcohol, acetone, oil, supercritical carbon dioxide, heat, pressure, pressure drop extraction, ultrasonic extraction, etc. Extracts can be a liquid, solid, dried liquid, re-suspended solid, etc.

### B. Amounts of Ingredients

It is contemplated that the compositions of the present invention can include any amount of the ingredients discussed in this specification. The compositions can also include any number of combinations of additional ingredients described throughout this specification (e.g., pigments, or additional cosmetic or pharmaceutical ingredients). The concentrations of the any ingredient within the compositions can vary. In non-limiting embodiments, for example, the compositions can comprise, consisting essentially of, or consist of, in their final form, for example, at least about 0.0001%, 0.0002%, 0.0003%, 0.0004%, 0.0005%, 0.0006%, 0.0007%, 0.0008%, 0.0009%, 0.0010%, 0.0011%, 0.0012%, 0.0013%, 0.0014%, 0.0015%, 0.0016%, 0.0017%, 0.0018%, 0.0019%, 0.0020%, 0.0021%, 0.0022%, 0.0023%, 0.0024%, 0.0025%, 0.0026%, 0.0027%, 0.0028%, 0.0029%, 0.0030%, 0.0031%, 0.0032%, 0.0033%, 0.0034%, 0.0035%, 0.0036%, 0.0037%, 0.0038%, 0.0039%, 0.0040%, 0.0041%, 0.0042%, 0.0043%, 0.0044%, 0.0045%, 0.0046%, 0.0047%, 0.0048%, 0.0049%, 0.0050%, 0.0051%, 0.0052%, 0.0053%, 0.0054%, 0.0055%, 0.0056%, 0.0057%, 0.0058%, 0.0059%, 0.0060%, 0.0061%, 0.0062%, 0.0063%, 0.0064%, 0.0065%, 0.0066%, 0.0067%, 0.0068%, 0.0069%, 0.0070%, 0.0071%, 0.0072%, 0.0073%, 0.0074%, 0.0075%, 0.0076%, 0.0077%, 0.0078%, 0.0079%, 0.0080%, 0.0081%, 0.0082%, 0.0083%, 0.0084%, 0.0085%, 0.0086%, 0.0087%, 0.0088%, 0.0089%, 0.0090%, 0.0091%, 0.0092%, 0.0093%, 0.0094%, 0.0095%, 0.0096%, 0.0097%, 0.0098%, 0.0099%, 0.0100%, 0.0200%, 0.0250%, 0.0275%, 0.0300%, 0.0325%, 0.0350%, 0.0375%, 0.0400%, 0.0425%, 0.0450%, 0.0475%, 0.0500%, 0.0525%, 0.0550%, 0.0575%, 0.0600%, 0.0625%, 0.0650%, 0.0675%, 0.0700%, 0.0725%, 0.0750%, 0.0775%, 0.0800%, 0.0825%, 0.0850%, 0.0875%, 0.0900%, 0.0925%, 0.0950%, 0.0975%, 0.1000%, 0.1250%, 0.1500%, 0.1750%, 0.2000%, 0.2250%, 0.2500%, 0.2750%, 0.3000%, 0.3250%, 0.3500%, 0.3750%, 0.4000%, 0.4250%, 0.4500%, 0.4750%, 0.5000%, 0.5250%, 0.0550%, 0.5750%, 0.6000%, 0.6250%, 0.6500%, 0.6750%, 0.7000%, 0.7250%, 0.7500%, 0.7750%, 0.8000%, 0.8250%, 0.8500%, 0.8750%, 0.9000%, 0.9250%, 0.9500%, 0.9750%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 60%, 65%, 70%, 75%, 80%, 85%, 90%, 95%, or 99% or any range derivable therein, of at least one of the ingredients that are mentioned throughout the specification and claims. In non-limiting aspects, the percentage can be calculated by weight or volume of the total composition. A person of ordinary skill in the art would understand that the concentrations can vary depending on the addition, substitution, and/or subtraction of ingredients in a given composition.

### C. Vehicles

The compositions of the present invention can include or be incorporated into all types of vehicles and carriers. The vehicle or carrier can be a pharmaceutically or dermatologically acceptable vehicle or carrier. Non-limiting examples of vehicles or carriers include water, glycerin, alcohol, oil, a silicon containing compound, a silicone compound, and wax. Variations and other appropriate vehicles will be apparent to the skilled artisan and are appropriate for use in the present invention. In certain aspects, the concentrations and combinations of the compounds, ingredients, and agents can be selected in such a way that the combinations are chemically compatible and do not form complexes which precipitate from the finished product.

### D. Structure

The compositions of the present invention can be structured or formulated into a variety of different forms. Non-limiting examples include emulsions (e.g., water-in-oil, water-in-oil-in-water, oil-in-water, silicone-in-water, water-in-silicone, oil-in-water-in-oil, oil-in-water-in-silicone emulsions), creams, lotions, solutions (both aqueous and hydro-alcoholic), anhydrous bases (such as lipsticks and powders), gels, masks, scrubs, body butters, peels, and ointments. Variations and other structures will be apparent to the skilled artisan and are appropriate for use in the present invention.

### E. Additional Ingredients

In addition to the combination of ingredients disclosed by the inventors, the compositions can also include additional ingredients such as cosmetic ingredients and pharmaceutical active ingredients. Non-limiting examples of these additional ingredients are described in the following subsections.

### 1. Cosmetic Ingredients

The CTFA International Cosmetic Ingredient Dictionary and Handbook (2004 and 2008) describes a wide variety of non-limiting cosmetic ingredients that can be used in the context of the present invention. Examples of these ingredient classes include: fragrance agents (artificial and natural; e.g., gluconic acid, phenoxyethanol, and triethanolamine), dyes and color ingredients (e.g., Blue 1, Blue 1 Lake, Red 40, titanium dioxide, D&C blue no. 4, D&C green no. 5, D&C orange no. 4, D&C red no. 17, D&C red no. 33, D&C violet no. 2, D&C yellow no. 10, and D&C yellow no. 11), flavoring agents/aroma agents (e.g., Stevia rebaudiana (sweetleaf) extract, and menthol), adsorbents, lubricants, solvents, moisturizers (including, e.g., emollients, humectants, film formers, occlusive agents, and agents that affect the natural moisturization mechanisms of the skin), water-repellants, UV absorbers (physical and chemical absorbers such as para-aminobenzoic acid (“PABA”) and corresponding PABA derivatives, titanium dioxide, zinc oxide, etc.), essential oils, vitamins (e.g., A, B, C, D, E, and K), trace metals (e.g., zinc, calcium and selenium), anti-irritants (e.g., steroids and non-steroidal anti-inflammatories), botanical extracts (e.g., Aloe vera, chamomile, cucumber extract, Ginkgo biloba, ginseng, and rosemary), anti-microbial agents, antioxidants (e.g., BHT and tocopherol), chelating agents (e.g., disodium EDTA and tetrasodium EDTA), preservatives (e.g., methylparaben and propylparaben), pH adjusters (e.g., sodium hydroxide and citric acid), absorbents (e.g., aluminum starch octenylsuccinate, kaolin, corn starch, oat starch, cyclodextrin, talc, and zeolite), skin bleaching and lightening agents (e.g., hydroquinone and niacinamide lactate), humectants (e.g., sorbitol, urea, methyl gluceth-20, saccharide isomerate, and mannitol), exfoliants, waterproofing agents (e.g., magnesium/aluminum hydroxide stearate), skin conditioning agents (e.g., aloe extracts, allantoin, bisabolol, ceramides, dimethicone, hyaluronic acid, biosaccharide gum-1, ethylhexylglycerin, pentylene glycol, hydrogenated polydecene, octyldodecyl oleate, and dipotassium glycyrrhizate). Non-limiting examples of some of these ingredients are provided in the following subsections.

a. UV Absorption and/or Reflecting Agents

UV absorption and/or reflecting agents that can be used in combination with the compositions of the present invention include chemical and physical sunblocks. Non-limiting examples of chemical sunblocks that can be used include para-aminobenzoic acid (PABA), PABA esters (glyceryl PABA, amyldimethyl PABA and octyldimethyl PABA), butyl PABA, ethyl PABA, ethyl dihydroxypropyl PABA, benzophenones (oxybenzone, sulisobenzone, benzophenone, and benzophenone-1 through 12), cinnamates (octyl methoxycinnamate (octinoxate), isoamyl p-methoxycinnamate, octylmethoxy cinnamate, cinoxate, diisopropyl methyl cinnamate, DEA-methoxycinnamate, ethyl diisopropylcinnamate, glyceryl octanoate dimethoxycinnamate and ethyl methoxycinnamate), cinnamate esters, salicylates (homomethyl salicylate, benzyl salicylate, glycol salicylate, isopropylbenzyl salicylate, etc.), anthranilates, ethyl urocanate, homosalate, octisalate, dibenzoylmethane derivatives (e.g., avobenzone), octocrylene, octyl triazone, digalloyl trioleate, glyceryl aminobenzoate, lawsone with dihydroxyacetone, ethylhexyl triazone, dioctyl butamido triazone, benzylidene malonate polysiloxane, terephthalylidene dicamphor sulfonic acid, disodium phenyl dibenzimidazole tetrasulfonate, diethylamino hydroxybenzoyl hexyl benzoate, bis diethylamino hydroxybenzoyl benzoate, bis benzoxazoylphenyl ethylhexylimino triazine, drometrizole trisiloxane, methylene bis-benzotriazolyl tetramethylbutylphenol, and bis-ethylhexyloxyphenol methoxyphenyltriazine, 4-methylbenzylidene camphor, and isopentyl 4-methoxycinnamate. Non-limiting examples of physical sunblocks include, kaolin, talc, petrolatum and metal oxides (e.g., titanium dioxide and zinc oxide).

b. Moisturizing Agents

Non-limiting examples of moisturizing agents that can be used with the compositions of the present invention include amino acids, chondroitin sulfate, diglycerin, erythritol, fructose, glucose, glycerin, glycerol polymers, glycol, 1,2,6-hexanetriol, honey, hyaluronic acid, hydrogenated honey, hydrogenated starch hydrolysate, inositol, lactitol, maltitol, maltose, mannitol, natural moisturizing factor, PEG-15 butanediol, polyglyceryl sorbitol, salts of pyrrolidone carboxylic acid, potassium PCA, propylene glycol, saccharide isomerate, sodium glucuronate, sodium PCA, sorbitol, sucrose, trehalose, urea, and xylitol.

Other examples include acetylated lanolin, acetylated lanolin alcohol, alanine, algae extract, Aloe barbadensis, Aloe barbadensis extract, Aloe barbadensis gel, Althea officinalis extract, apricot (Prunus armeniaca) kernel oil, arginine, arginine aspartate, Arnica montana extract, aspartic acid, avocado (Persea gratissima) oil, barrier sphingolipids, butyl alcohol, beeswax, behenyl alcohol, beta-sitosterol, birch (Betula alba) bark extract, borage (Borago officinalis) extract, butcherbroom (Ruscus aculeatus) extract, butylene glycol, Calendula officinalis extract, Calendula officinalis oil, candelilla (Euphorbia cerifera) wax, canola oil, caprylic/capric triglyceride, cardamom (Elettaria cardamomum) oil, carnauba (Copernicia cerifera) wax, carrot (Daucus carota sativa) oil, castor (Ricinus communis) oil, ceramides, ceresin, ceteareth-5, ceteareth-12, ceteareth-20, cetearyl octanoate, ceteth-20, ceteth-24, cetyl acetate, cetyl octanoate, cetyl palmitate, chamomile (Anthemis nobilis) oil, cholesterol, cholesterol esters, cholesteryl hydroxystearate, citric acid, clary (Salvia sclarea) oil, cocoa (Theobroma cacao) butter, coco-caprylate/caprate, coconut (Cocos nucifera) oil, collagen, collagen amino acids, corn (Zea mays) oil, fatty acids, decyl oleate, dimethicone copolyol, dimethiconol, adipate, dioctyl dioctyl succinate, dipentaerythrityl hexacaprylate/hexacaprate, DNA, erythritol, ethoxydiglycol, ethyl linoleate, Eucalyptus globulus oil, evening primrose (Oenothera biennis) oil, fatty acids, Geranium maculatum oil, glucosamine, glucose glutamate, glutamic acid, glycereth-26, glycerin, glycerol, glyceryl distearate, glyceryl hydroxystearate, glyceryl laurate, glyceryl linoleate, glyceryl myristate, glyceryl oleate, glyceryl stearate, glyceryl stearate SE, glycine, glycol stearate, glycol stearate SE, glycosaminoglycans, grape (Vitis vinifera) seed oil, hazel (Corylus americana) nut oil, hazel (Corylus avellana) nut oil, hexylene glycol, hyaluronic acid, hybrid safflower (Carthamus tinctorius) oil, hydrogenated castor oil, hydrogenated coco-glycerides, hydrogenated coconut oil, hydrogenated lanolin, hydrogenated lecithin, hydrogenated palm glyceride, hydrogenated palm kernel oil, hydrogenated soybean oil, hydrogenated tallow glyceride, hydrogenated vegetable oil, hydrolyzed collagen, hydrolyzed elastin, hydrolyzed glycosaminoglycans, hydrolyzed keratin, hydrolyzed soy protein, hydroxylated lanolin, hydroxyproline, isocetyl stearate, isocetyl stearoyl stearate, isodecyl oleate, isopropyl isostearate, isopropyl lanolate, isopropyl myristate, isopropyl palmitate, isopropyl stearate, isostearamide DEA, isostearic acid, isostearyl lactate, isostearyl neopentanoate, jasmine (Jasminum officinale) oil, jojoba (Buxus chinensis) oil, kelp, kukui (Aleurites moluccana) nut oil, lactamide MEA, laneth-16, laneth-10 acetate, lanolin, lanolin acid, lanolin alcohol, lanolin oil, lanolin wax, lavender (Lavandula angustifolia) oil, lecithin, lemon (Citrus medica limonum) oil, linoleic acid, linolenic acid, Macadamia ternifolia nut oil, maltitol, matricaria (Chamomilla recutita) oil, methyl glucose sesquistearate, methylsilanol PCA, mineral oil, mink oil, mortierella oil, myristyl lactate, myristyl myristate, myristyl propionate, neopentyl glycol dicaprylate/dicaprate, octyldodecanol, octyldodecyl myristate, octyldodecyl stearoyl stearate, octyl hydroxystearate, octyl palmitate, octyl salicylate, octyl stearate, oleic acid, olive (Olea europaea) oil, orange (Citrus aurantium dulcis) oil, palm (Elaeis guineensis) oil, palmitic acid, pantethine, panthenol, panthenyl ethyl ether, paraffin, PCA, peach (Prunus persica) kernel oil, peanut (Arachis hypogaea) oil, PEG-8 C12-18 ester, PEG-15 cocamine, PEG-150 distearate, PEG-60 glyceryl isostearate, PEG-5 glyceryl stearate, PEG-30 glyceryl stearate, PEG-7 hydrogenated castor oil, PEG-40 hydrogenated castor oil, PEG-60 hydrogenated castor oil, PEG-20 methyl glucose sesquistearate, PEG-40 sorbitan peroleate, PEG-5 soy sterol, PEG-10 soy sterol, PEG-2 stearate, PEG-8 stearate, PEG-20 stearate, PEG-32 stearate, PEG-40 stearate, PEG-50 stearate, PEG-100 stearate, PEG-150 stearate, pentadecalactone, peppermint (Mentha piperita) oil, petrolatum, phospholipids, plankton extract, polyamino sugar condensate, polyglyceryl-3 diisostearate, polyquaternium-24, polysorbate 20, polysorbate 40, polysorbate 60, polysorbate 80, polysorbate 85, potassium myristate, potassium palmitate, propylene glycol, propylene glycol dicaprylate/dicaprate, propylene glycol dioctanoate, propylene glycol dipelargonate, propylene glycol laurate, propylene glycol stearate, propylene glycol stearate SE, PVP, pyridoxine dipalmitate, retinol, retinyl palmitate, rice (Oryza sativa) bran oil, RNA, rosemary (Rosmarinus officinalis) oil, rose oil, safflower (Carthamus tinctorius) oil, sage (Salvia officinalis) oil, sandalwood (Santalum album) oil, serine, serum protein, sesame (Sesamum indicum) oil, shea butter (Butyrospermum parkii), silk powder, sodium chondroitin sulfate, sodium hyaluronate, sodium lactate, sodium palmitate, sodium PCA, sodium polyglutamate, soluble collagen, sorbitan laurate, sorbitan oleate, sorbitan palmitate, sorbitan sesquioleate, sorbitan stearate, sorbitol, soybean (Glycine soja) oil, sphingolipids, squalane, squalene, stearamide MEA-stearate, acid, stearic stearoxy dimethicone, stearoxytrimethylsilane, stearyl alcohol, stearyl glycyrrhetinate, stearyl heptanoate, stearyl stearate, sunflower (Helianthus annuus) seed oil, sweet almond (Prunus amygdalus dulcis) oil, synthetic beeswax, tocopherol, tocopheryl acetate, tocopheryl linoleate, tribehenin, tridecyl neopentanoate, tridecyl stearate, triethanolamine, tristearin, urea, vegetable oil, water, waxes, wheat (Triticum vulgare) germ oil, and ylang ylang (Cananga odorata) oil.

c. Antioxidants

Non-limiting examples of antioxidants that can be used with the compositions of the present invention include acetyl cysteine, ascorbic acid polypeptide, ascorbyl dipalmitate, ascorbyl methylsilanol pectinate, ascorbyl palmitate, ascorbyl stearate, BHA, BHT, t-butyl hydroquinone, cysteine, cysteine HCl, diamylhydroquinone, di-t-butylhydroquinone, dicetyl thiodipropionate, dioleyl tocopheryl methylsilanol, disodium ascorbyl sulfate, distearyl thiodipropionate, ditridecyl thiodipropionate, dodecyl gallate, erythorbic acid, esters of ascorbic acid, ethyl ferulate, ferulic acid, gallic acid esters, hydroquinone, isooctyl thioglycolate, kojic acid, magnesium ascorbate, magnesium ascorbyl phosphate, methylsilanol ascorbate, natural botanical anti-oxidants such as green tea or grape seed extracts, nordihydroguaiaretic acid, octyl gallate, phenylthioglycolic acid, potassium ascorbyl tocopheryl phosphate, potassium sulfite, propyl gallate, quinones, rosmarinic acid, sodium ascorbate, sodium bisulfite, sodium erythorbate, sodium metabisulfite, sodium sulfite, superoxide dismutase, sodium thioglycolate, sorbityl furfural, thiodiglycol, thiodiglycolamide, thiodiglycolic acid, thioglycolic acid, thiolactic acid, thiosalicylic acid, tocophereth-5, tocophereth-10, tocophereth-12, tocophereth-18, tocophereth-50, tocopherol, tocophersolan, tocopheryl acetate, tocopheryl linoleate, tocopheryl nicotinate, tocopheryl succinate, and tris(nonylphenyl)phosphite.

d. Structuring Agents

In other non-limiting aspects, the compositions of the present invention can include a structuring agent. Structuring agent, in certain aspects, assist in providing rheological characteristics to the composition to contribute to the composition's stability. In other aspects, structuring agents can also function as an emulsifier or surfactant. Non-limiting examples of structuring agents include stearic acid, palmitic acid, stearyl alcohol, cetyl alcohol, behenyl alcohol, stearic acid, palmitic acid, the polyethylene glycol ether of stearyl alcohol having an average of about 1 to about 21 ethylene oxide units, the polyethylene glycol ether of cetyl alcohol having an average of about 1 to about 5 ethylene oxide units, and mixtures thereof.

e. Emulsifiers

In certain aspects of the present invention, the compositions do not include an emulsifier. In other aspects, however, the compositions can include one or more emulsifiers. Emulsifiers can reduce the interfacial tension between phases and improve the formulation and stability of an emulsion. The emulsifiers can be nonionic, cationic, anionic, and zwitterionic emulsifiers (see U.S. Pat. Nos. 5,011,681; 4,421,769; 3,755,560). Non-limiting examples include esters of glycerin, esters of propylene glycol, fatty acid esters of polyethylene glycol, fatty acid esters of polypropylene glycol, esters of sorbitol, esters of sorbitan anhydrides, carboxylic acid copolymers, esters and ethers of glucose, ethoxylated ethers, ethoxylated alcohols, alkyl phosphates, polyoxyethylene fatty ether phosphates, fatty acid amides, acyl lactylates, soaps, TEA stearate, DEA oleth-3 phosphate, polyethylene glycol 20 sorbitan monolaurate (polysorbate 20), polyethylene glycol 5 soya sterol, steareth-2, steareth-20, steareth-21, ceteareth-20, cetearyl glucoside, cetearyl alcohol, C12-13 pareth-3, PPG-2 methyl glucose ether distearate, PPG-5-ceteth-20, bis-PEG/PPG-20/20 dimethicone, ceteth-10, polysorbate 80, cetyl phosphate, potassium cetyl phosphate, diethanolamine cetyl phosphate, polysorbate 60, glyceryl stearate, PEG-100 stearate, arachidyl alcohol, arachidyl glucoside, and mixtures thereof.

f. Silicone Containing Compounds

In non-limiting aspects, silicone containing compounds include any member of a family of polymeric products whose molecular backbone is made up of alternating silicon and oxygen atoms with side groups attached to the silicon atoms. By varying the —Si—O— chain lengths, side groups, and crosslinking, silicones can be synthesized into a wide variety of materials. They can vary in consistency from liquid to gel to solids.

The silicone containing compounds that can be used in the context of the present invention include those described in this specification or those known to a person of ordinary skill in the art. Non-limiting examples include silicone oils (e.g., volatile and non-volatile oils), gels, and solids. In certain aspects, the silicon containing compounds include silicone oils such as a polyorganosiloxane. Non-limiting examples of polyorganosiloxanes include dimethicone, cyclomethicone, polysilicone-11, phenyl trimethicone, trimethylsilylamodimethicone, stearoxytrimethylsilane, or mixtures of these and other organosiloxane materials in any given ratio in order to achieve the desired consistency and application characteristics depending upon the intended application (e.g., to a particular area such as the skin, hair, or eyes). A “volatile silicone oil” includes a silicone oil have a low heat of vaporization, i.e. normally less than about 50 cal per gram of silicone oil. Non-limiting examples of volatile silicone oils include: cyclomethicones such as Dow Corning 344 Fluid, Dow Corning 345 Fluid, Dow Corning 244 Fluid, and Dow Corning 245 Fluid, Volatile Silicon 7207 (Union Carbide Corp., Danbury, Conn.); low viscosity dimethicones, i.e. dimethicones having a viscosity of about 50 cst or less (e.g., dimethicones such as Dow Corning 200-0.5 cst Fluid). The Dow Corning Fluids are available from Dow Corning Corporation, Midland, Michigan. Cyclomethicone and dimethicone are described in the Third Edition of the CTFA Cosmetic Ingredient Dictionary (incorporated by reference) as cyclic dimethyl polysiloxane compounds and a mixture of fully methylated linear siloxane polymers end-blocked with trimethylsiloxy units, respectively. Other non-limiting volatile silicone oils that can be used in the context of the present invention include those available from General Electric Co., Silicone Products Div., Waterford, N.Y. and SWS Silicones Div. of Stauffer Chemical Co., Adrian, Michigan.

g. Exfoliating Agent

Exfoliating agents include ingredients that remove dead skin cells on the skin's outer surface. These agents may act through mechanical, chemical, and/or other means. Non-limiting examples of mechanical exfoliating agents include abrasives such as pumice, silica, cloth, paper, shells, beads, solid crystals, solid polymers, etc. Non-limiting examples of chemical exfoliating agents include acids and enzyme exfoliants. Acids that can be used as exfoliating agents include, but are not limited to, glycolic acid, lactic acid, citric acid, alpha hydroxy acids, beta hydroxy acids, etc. Other exfoliating agents known to those of skill in the art are also contemplated as being useful within the context of the present invention.

h. Essential Oils

Essential oils include oils derived from herbs, flowers, trees, and other plants. Such oils are typically present as tiny droplets between the plant's cells, and can be extracted by several method known to those of skill in the art (e.g., steam distilled, enfleurage (i.e., extraction by using fat), maceration, solvent extraction, or mechanical pressing). When these types of oils are exposed to air they tend to evaporate (i.e., a volatile oil). As a result, many essential oils are colorless, but with age they can oxidize and become darker. Essential oils are insoluble in water and are soluble in alcohol, ether, fixed oils (vegetal), and other organic solvents. Typical physical characteristics found in essential oils include boiling points that vary from about 160° to 240° C. and densities ranging from about 0.759 to about 1.096.

Essential oils typically are named by the plant from which the oil is found. For example, rose oil or peppermint oil are derived from rose or peppermint plants, respectively. Non-limiting examples of essential oils that can be used in the context of the present invention include sesame oil, macadamia nut oil, tea tree oil, evening primrose oil, Spanish sage oil, Spanish rosemary oil, coriander oil, thyme oil, pimento berries oil, rose oil, anise oil, balsam oil, bergamot oil, rosewood oil, cedar oil, chamomile oil, sage oil, clary sage oil, clove oil, cypress oil, eucalyptus oil, fennel oil, sea fennel oil, frankincense oil, geranium oil, ginger oil, grapefruit oil, jasmine oil, juniper oil, lavender oil, lemon oil, lemongrass oil, lime oil, mandarin oil, marjoram oil, myrrh oil, neroli oil, orange oil, patchouli oil, pepper oil, black pepper oil, petitgrain oil, pine oil, rose otto oil, rosemary oil, sandalwood oil, spearmint oil, spikenard oil, vetiver oil, wintergreen oil, or ylang-ylang. Other essential oils known to those of skill in the art are also contemplated as being useful within the context of the present invention.

i. Thickening Agents

Thickening agents, including thickener or gelling agents, include substances which that can increase the viscosity of a composition. Thickeners include those that can increase the viscosity of a composition without substantially modifying the efficacy of the active ingredient within the composition. Thickeners can also increase the stability of the compositions of the present invention. In certain aspects of the present invention, thickeners include hydrogenated polyisobutene, trihydroxystearin, ammonium acryloyldimethyltaurate/VP copolymer, or a mixture of them.

Non-limiting examples of additional thickening agents that can be used in the context of the present invention include carboxylic acid polymers, crosslinked polyacrylate polymers, polyacrylamide polymers, polysaccharides, and gums. Examples of carboxylic acid polymers include crosslinked compounds containing one or more monomers derived from acrylic acid, substituted acrylic acids, and salts and esters of these acrylic acids and the substituted acrylic acids, wherein the crosslinking agent contains two or more carbon-carbon double bonds and is derived from a polyhydric alcohol (see U.S. Pat. Nos. 5,087,445; 4,509,949; 2,798,053; CTFA International Cosmetic Ingredient Dictionary, Fourth edition, 1991, pp. 12 and 80). Examples of commercially available carboxylic acid polymers include carbomers, which are homopolymers of acrylic acid crosslinked with allyl ethers of sucrose or pentaerythritol (e.g., CARBOPOL™ 900 series from B. F. GOODRICH).

Non-limiting examples of crosslinked polyacrylate polymers include cationic and nonionic polymers. Examples are described in U.S. Pat. Nos. 5,100,660; 4,849,484; 4,835,206; 4,628,078; 4,599,379).

Non-limiting examples of polyacrylamide polymers (including nonionic polyacrylamide polymers including substituted branched or unbranched polymers) include polyacrylamide, isoparaffin and laureth-7, multi-block copolymers of acrylamides and substituted acrylamides with acrylic acids and substituted acrylic acids.

Non-limiting examples of polysaccharides include cellulose, carboxymethyl hydroxyethylcellulose, cellulose acetate propionate carboxylate, hydroxyethylcellulose, hydroxyethyl ethylcellulose, hydroxypropylcellulose, hydroxypropyl methylcellulose, methyl hydroxyethylcellulose, microcrystalline cellulose, sodium cellulose sulfate, and mixtures thereof. Another example is an alkyl substituted cellulose where the hydroxy groups of the cellulose polymer is hydroxyalkylated (preferably hydroxy ethylated or hydroxypropylated) to form a hydroxyalkylated cellulose which is then further modified with a C10-C30 straight chain or branched chain alkyl group through an ether linkage. Typically these polymers are ethers of C10-C30 straight or branched chain alcohols with hydroxyalkylcelluloses. Other useful polysaccharides include scleroglucans comprising a linear chain of (1-3) linked glucose units with a (1-6) linked glucose every three units.

Non-limiting examples of gums that can be used with the present invention include acacia, agar, algin, alginic acid, ammonium alginate, amylopectin, calcium alginate, calcium carrageenan, carnitine, carrageenan, dextrin, gelatin, gellan gum, guar gum, guar hydroxypropyltrimonium chloride, hectorite, hyaluronic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboxymethyl dextran, sodium carrageenan, tragacanth gum, xanthan gum, and mixtures thereof.

j. Preservatives

Non-limiting examples of preservatives that can be used in the context of the present invention include quaternary ammonium preservatives such as polyquaternium-1 and benzalkonium halides (e.g., benzalkonium chloride (“BAC”) and benzalkonium bromide), parabens (e.g., methylparabens and propylparabens), phenoxyethanol, benzyl alcohol, chlorobutanol, phenol, sorbic acid, thimerosal or combinations thereof.

### 2. Pharmaceutical Ingredients

Pharmaceutical active agents are also contemplated as being useful with the compositions of the present invention. Non-limiting examples of pharmaceutical active agents include anti-acne agents, agents used to treat rosacea, analgesics, anesthetics, anorectals, antihistamines, anti-inflammatory agents including non-steroidal anti-inflammatory drugs, antibiotics, antifungals, antivirals, antimicrobials, anti-cancer actives, scabicides, pediculicides, antineoplastics, antiperspirants, antipruritics, antipsoriatic agents, antiseborrheic agents, biologically active proteins and peptides, burn treatment agents, cauterizing agents, depigmenting agents, depilatories, diaper rash treatment agents, enzymes, hair growth stimulants, hair growth retardants including DFMO and its salts and analogs, hemostatics, kerotolytics, canker sore treatment agents, cold sore treatment agents, dental and periodontal treatment agents, photosensitizing actives, skin protectant/barrier agents, steroids including hormones and corticosteroids, sunburn treatment agents, sunscreens, transdermal actives, nasal actives, vaginal actives, wart treatment agents, wound treatment agents, wound healing agents, etc.

### F. Kits

Kits are also contemplated as being used in certain aspects of the present invention. For instance, compositions of the present invention can be included in a kit. A kit can include a container. Containers can include a bottle, a metal tube, a laminate tube, a plastic tube, a dispenser, a pressurized container, a barrier container, a package, a compartment, a lipstick container, a compact container, cosmetic pans that can hold cosmetic compositions, or other types of containers such as injection or blow-molded plastic containers into which the dispersions or compositions or desired bottles, dispensers, or packages are retained. The kit and/or container can include indicia on its surface. The indicia, for example, can be a word, a phrase, an abbreviation, a picture, or a symbol.

The containers can dispense a pre-determined amount of the composition. In other embodiments, the container can be squeezed (e.g., metal, laminate, or plastic tube) to dispense a desired amount of the composition. The composition can be dispensed as a spray, an aerosol, a liquid, a fluid, or a semi-solid. The containers can have spray, pump, or squeeze mechanisms. A kit can also include instructions for employing the kit components as well the use of any other compositions included in the container. Instructions can include an explanation of how to apply, use, and maintain the compositions.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### Example 1

### Exemplary Formulations

Formulations having the ingredients from Example 1 were prepared as topical skin compositions. The formulations in Tables 1 to 4 are examples of topical skin compositions prepared as a luminous cosmetic composition and mattifying cosmetic composition respectively.

TABLE 1*

| | Luminous Composition | | | | |
|---|---|---|---|---|---|
| | AB1700 | AB1703 | AB1705 | AB1710 | AB1734 |
| Ingredient | % wt | % wt | % wt | % wt | % wt |
| Water | q.s. | q.s. | q.s. | q.s. | q.s. |
| Dimethicone | 8.19 | 8.18 | 7.98 | 8.17 | 8.17 |
| Butylene glycol | 8.00 | 8.00 | 8.00 | 8.00 | 8.00 |
| Glycerin | 6.68 | 6.68 | 6.68 | 6.68 | 6.68 |
| Neopentyl glycol | 5.39 | 5.04 | 5.44 | 5.05 | 6.09 |
| diheptanoate | | | | | |
| Phenylisopropyl | 4.98 | 4.67 | 5.03 | 4.68 | 5.63 |
| dimethicone | | | | | |
| Methyl trimethicone | 3.00 | 3.00 | 3.00 | 3.00 | 3.00 |
| Lauryl PEG-9 | 2.50 | 2.50 | 2.50 | 2.50 | 2.50 |
| polydimethylsiloxyethyl | | | | | |
| Dimethicone | | | | | |
| Acrylates/dimethicone | 2.00 | 2.00 | 2.00 | 2.00 | 2.00 |
| copolymer | | | | | |
| Dimethicone/PEG-10/15 | 1.20 | 1.20 | 1.20 | 1.20 | 1.20 |
| crosspolymer | | | | | |
| Boron nitride | 0.00 | 0.00 | 0.00 | 1.00 | 1.00 |
| Lauroyl lysine | 0.00 | 1.00 | 0.00 | 0.00 | 0.00 |
| Silica | 0.00 | 0.00 | 1.00 | 0.00 | 0.00 |
| Magnesium sulfate | 1.00 | 1.00 | 1.00 | 1.00 | 1.00 |
| Niacinamide | 1.00 | 1.00 | 1.00 | 1.00 | 1.00 |
| PEG-9 dimethicone | 1.00 | 1.00 | 1.00 | 1.00 | 1.00 |
| Jojoba wax | 0.60 | 0.60 | 0.60 | 0.60 | 0.60 |
| PEG-120 esters | | | | | |
| Titanium dioxide | 11.06 | 10.94 | 10.40 | 10.94 | 10.94 |
| Iron oxides | 0.59 | 0.54 | 0.54 | 0.52 | 0.52 |
| Aluminum hydroxide | 0.36 | 0.36 | 0.34 | 0.36 | 0.36 |
| Caprylyl glycol | 0.20 | 0.20 | 0.17 | 0.17 | 0.17 |
| Chlorphenesin | 0.16 | 0.16 | 0.14 | 0.14 | 0.14 |
| Mica | 0.15 | 0.00 | 0.00 | 0.00 | 0.00 |
| Disodium stearoyl | 0.13 | 0.13 | 0.12 | 0.13 | 0.13 |
| glutamate | | | | | |
| Tocopheryl acetate | 0.10 | 0.10 | 0.10 | 0.10 | 0.10 |
| Disodium EDTA | 0.05 | 0.05 | 0.05 | 0.05 | 0.05 |
| Isopropyl titanium | 10.01 | 0.00 | 0.00 | 0.00 | 0.00 |
| triisostearate | | | | | |
| Alcohol | 0.01 | 0.01 | 0.01 | 0.01 | 0.01 |
| Excipients** | q.s. | q.s. | q.s. | q.s. | q.s. |

*Formulation can be prepared by mixing the ingredients in a beaker under heat 70-75° C. until homogenous. Subsequently, the formulation can be cooled to standing room temperature (20-25° C.). Further, and if desired, additional ingredients can be added, for example, to modify the rheological properties of the composition.
**Excipients can be added, for example, to modify the rheological properties of the composition. Alternatively, the amount of water can be varied so long as the amount of water in the composition is at least 35% w/w, and preferably between 35 to 55% w/w.

TABLE 2*

| | Luminous Composition | |
|---|---|---|
| | 70215263007 | 70215263010 |
| Ingredient | % wt | % wt |
| Water/eau | 37.94842 | 37.98290 |
| Dimethicone | 14.00008 | 14.00018 |
| Titanium dioxide | 12.62616 | 12.63418 |
| Butylene glycol | 11.70000 | 11.70000 |
| Glycerin | 6.73864 | 6.70367 |
| Cetyl PEG/PPG-10/1 dimethicone | 3.99980 | 3.99863 |
| Pentaerythrityl tetraisostearate | 3.80000 | 3.80000 |
| PEG-9 dimethicone | 2.00000 | 2.00000 |
| Boron nitride | 1.00000 | 1.00000 |
| Jojoba wax PEG-120 esters | 1.00000 | 1.00000 |
| Magnesium sulfate | 1.00000 | 1.00000 |
| Niacinamide | 1.00000 | 1.00000 |
| Iron oxides | 0.61243 | 0.60407 |
| Trimethylsiloxysilicate | 0.60000 | 0.60000 |
| Phenoxyethanol | 0.54420 | 0.54420 |
| Aluminum hydroxide | 0.41528 | 0.41553 |
| Methyl trimethicone | 0.40000 | 0.40000 |
| Caprylyl glycol | 0.17000 | 0.17000 |
| Disodium stearoyl glutamate | 0.15004 | 0.15004 |
| Chlorphenesin | 0.13600 | 0.13600 |
| Tocopheryl acetate | 0.10012 | 0.10012 |
| Disodium EDTA | 0.05000 | 0.05000 |
| Sucrose stearate | 0.00433 | 0.00433 |
| Alcohol denat. | 0.00162 | 0.00162 |
| Dipropylene glycol | 0.00108 | 0.00108 |
| Ethyl macadamiate | 0.00108 | 0.00108 |
| Resveratrol | 0.00065 | 0.00065 |
| Oligopeptide-1 | 0.00006 | 0.00005 |
| Pentaerythrityl tetradit-butyl | 0.00000 | 0.00093 |
| hydroxyhydrocinnamate | | |
| Triisopropanolamine | 0.00000 | 0.00044 |
| Xanthan gum | 0.00000 | 0.00012 |
| Citric acid | 0.00000 | 0.00006 |
| Caproic acid | 0.00000 | 0.00005 |
| Dextran | 0.00000 | 0.00005 |
| Potassium sorbate | 0.00000 | 0.00002 |
| Malic acid | 0.00000 | 0.00000 |
| Tocopherol | 0.00000 | 0.00000 |
| Excipients** | q.s. | q.s. |

*Formulation can be prepared by mixing the ingredients in a beaker under heat 70-75° C. until homogenous. Subsequently, the formulation can be cooled to standing room temperature (20-25° C.). Further, and if desired, additional ingredients can be added, for example, to modify the rheological properties of the composition.
**Excipients can be added, for example, to modify the rheological properties of the composition. Alternatively, the amount of water can be varied so long as the amount of water in the composition is at least 35% w/w, and preferably between 35 to 55% w/w.

TABLE 3*

| | Mattifying Composition | | | | |
|---|---|---|---|---|---|
| | AA3682 | AA3693 | AA3699 | AB2804 | AB2805 |
| Ingredient | % wt | % wt | % wt | % wt | % wt |
| Water | q.s. | q.s. | q.s. | q.s. | q.s. |
| Titanium dioxide | 9.48 | 9.48 | 9.48 | 9.48 | 9.48 |
| Dimethicone | 8.59 | 8.59 | 8.59 | 8.59 | 8.59 |
| Methyl | 7 | 7 | 4.8 | 5.4 | 6 |
| trimethicone | | | | | |
| (60% of film | | | | | |
| former + free) | | | | | |
| Isododecane | 4 | 4 | 6 | 6 | 6 |
| Silica | 3.75 | 0 | 3.75 | 3.75 | 1.75 |
| Butylene glycol | 3.5 | 3.5 | 3.5 | 3.5 | 3.5 |
| Lauryl PEG-9 | 2.5 | 2.5 | 2.5 | 2.5 | 2.5 |
| polydimethyl- | | | | | |
| siloxyethyl | | | | | |
| dimethicone | | | | | |
| Iron oxides | 2.06 | 2.06 | 2.06 | 2.06 | 2.06 |
| Acrylates/ | 2 | 2 | 1.2 | 1.6 | 2 |
| dimethicone | | | | | |
| copolymer (40% | | | | | |
| of film former) | | | | | |
| Dimethicone/ | 1.2 | 1.2 | 1.2 | 1.2 | 1.2 |
| PEG-10/15 | | | | | |
| cross polymer | | | | | |
| Magnesium | 1 | 1 | 1 | 1 | 1 |
| sulfate | | | | | |
| Niacinamide | 1 | 1 | 1 | 1 | 1 |
| PEG-9 | 1 | 1 | 1 | 1 | 1 |
| dimethicone | | | | | |
| Cellulose | 1 | 3 | 1 | 1 | 2 |
| Phenoxyethanol | 0.54 | 0.54 | 0.54 | 0.54 | 0.54 |
| Aluminum | 0.31 | 0.31 | 0.31 | 0.31 | 0.31 |
| hydroxide | | | | | |
| Glycerin | 0.21 | 0.21 | 0.21 | 0.21 | 0.21 |
| Caprylyl glycol | 0.17 | 0.17 | 0.17 | 0.17 | 0.17 |
| Chlorphenesin | 0.14 | 0.14 | 0.14 | 0.14 | 0.14 |
| Disodium stearoyl | 0.13 | 0.13 | 0.13 | 0.13 | 0.13 |
| glutamate | | | | | |
| Tocopheryl | 0.1 | 0.1 | 0.1 | 0.1 | 0.1 |
| acetate | | | | | |
| Xanthan gum | 0.1 | 0.1 | 0.1 | 0.1 | 0.1 |
| Disodium EDTA | 0.05 | 0.05 | 0.05 | 0.05 | 0.05 |
| Dipropylene | 0.03 | 0.03 | 0.03 | 0.03 | 0.03 |
| glycol | | | | | |
| Sodium citrate | 0.01 | 0.01 | 0.01 | 0.01 | 0.01 |
| Sucrose stearate | 0.01 | 0.01 | 0.01 | 0.01 | 0.01 |
| Alcohol | 0.01 | 0.01 | 0.01 | 0.01 | 0.01 |
| Tocopherol | 0.01 | 0.01 | 0.01 | 0.01 | 0.01 |
| Ethyl | 0.01 | 0.01 | 0.01 | 0.01 | 0.01 |
| macadamiate | | | | | |
| Excipients** | q.s. | q.s. | q.s. | q.s. | q.s. |

*Formulation can be prepared by mixing the ingredients in a beaker under heat 70-75° C. until homogenous. Subsequently, the formulation can be cooled to standing room temperature (20-25° C.). Further, and if desired, additional ingredients can be added, for example, to modify the rheological properties of the composition.
**Excipients can be added, for example, to modify the rheological properties of the composition. Alternatively, the amount of water can be varied so long as the amount of water in the composition is at least 35% w/w, and preferably between approximately 35 to 55% w/w.

TABLE 4*
Mattifying Composition 70215420005

| Ingredient | % wt |
|---|---|
| Water/eau | 45.27910 |
| Titanium dioxide | 17.01404 |
| Dimethicone | 13.61380 |
| Silica | 3.75000 |
| Butylene glycol | 3.50000 |
| Lauryl PEG-9 polydimethylsiloxyethyl | 2.49950 |
| dimethicone | |
| Vp/hexadecene copolymer | 2.00000 |
| Trimethylsiloxysilicate | 1.59000 |
| Methyl trimethicone | 1.51000 |
| Dimethicone/PEG-10/15 crosspolymer | 1.20000 |
| Cetyl dimethicone | 1.00000 |
| Magnesium sulfate | 1.00000 |
| Niacinamide | 1.00000 |
| Pentaerythrityl tetraisostearate | 1.00000 |
| Zinc oxide | 1.00000 |
| Iron oxides | 0.62693 |
| Aluminum hydroxide | 0.55923 |
| Phenoxyethanol | 0.54420 |
| Acrylates/dimethicone copolymer | 0.30000 |
| Glycerin | 0.21144 |
| Disodium stearoyl glutamate | 0.20000 |
| Caprylyl glycol | 0.17000 |
| Chlorphenesin | 0.13600 |
| Tocopheryl acetate | 0.10012 |
| Xanthan gum | 0.10012 |
| Disodium EDTA | 0.05000 |
| Dipropylene glycol | 0.02608 |
| Sodium citrate | 0.01000 |
| Sucrose stearate | 0.00433 |
| Alcohol denat. | 0.00162 |
| Tocopherol | 0.00150 |
| Ethyl macadamiate | 0.00108 |
| Resveratrol | 0.00065 |
| Caproic acid | 0.00006 |
| Dextran | 0.00006 |
| Oligopeptide-1 | 0.00006 |
| Citric acid | 0.00006 |
| Potassium sorbate | 0.00002 |
| Malic acid | 0.00000 |
| Excipients** | q.s. |

*Formulation can be prepared by mixing the ingredients in a beaker under heat 70-75° C. until homogenous. Subsequently, the formulation can be cooled to standing room temperature (20-25° C.). Further, and if desired, additional ingredients can be added, for example, to modify the rheological properties of the composition.
**Excipients can be added, for example, to modify the rheological properties of the composition. Alternatively, the amount of water can be varied so long as the amount of water in the composition is at least 35% w/w, and preferably between approximately 35 to 55% w/w.

### Example 2

### Exemplary Assays

Assays that can be used to determine the efficacy of any one of the ingredients or any combination of ingredients or compositions having said combination of ingredients disclosed throughout the specification and claims can be determined by methods known to those of ordinary skill in the art. The following are non-limiting assays that can be used in the context of the present invention. It should be recognized that other testing procedures can be used, including, for example, objective and subjective procedures.

B16 Pigmentation Assay: Melanogenesis is the process by which melanocytes produce melanin, a naturally produced pigment that imparts color to skin, hair, and eyes. Inhibiting melanogenesis is beneficial to prevent skin darkening and lighten dark spots associated with aging. This bioassay utilizes B16-F1 melanocytes (ATCC), an immortalized mouse melanoma cell line, to analyze the effect of compounds on melanogenesis. The endpoint of this assay is a spectrophotometric measurement of melanin production and cellular viability. B16-F1 melanocytes, can be cultivated in standard DMEM growth medium with 10% fetal bovine serum (MEDIATECH) at 37° C. in 10% CO₂ and then treated with any one of the active ingredients, combination of ingredients, or compositions having said combinations disclosed in the specification for 6 days. Following incubation, melanin secretion is measured by absorbance at 405 nm and cellular viability is quantified.

Collagen Stimulation Assay: Collagen is an extracellular matrix protein critical for skin structure. Increased synthesis of collagen helps improve skin firmness and elasticity. This bioassay can be used to examine the effect of any one of the active ingredients, combination of ingredients, or compositions having said combinations disclosed in the specification on the production of procollagen peptide (a precursor to collagen) by human epidermal fibroblasts. The endpoint of this assay is a spectrophotometric measurement that reflects the presence of procollagen peptide and cellular viability. The assay employs the quantitative sandwich enzyme immunoassay technique whereby a monoclonal antibody specific for procollagen peptide has been pre-coated onto a microplate. Standards and samples can be pipetted into the wells and any procollagen peptide present is bound by the immobilized antibody. After washing away any unbound substances, an enzyme-linked polyclonal antibody specific for procollagen peptide can be added to the wells. Following a wash to remove any unbound antibody-enzyme reagent, a substrate solution can be added to the wells and color develops in proportion to the amount of procollagen peptide bound in the initial step using a microplate reader for detection at 450 nm. The color development can be stopped and the intensity of the color can be measured.

For generation of samples and controls, subconfluent normal human adult epidermal fibroblasts (Cascade Biologics) cultivated in standard DMEM growth medium with 10% fetal bovine serum (MEDIATECH) at 37° C. in 10% CO₂, can be treated with each of the combination of ingredients or compositions having said combinations disclosed in the specification for 3 days. Following incubation, cell culture medium can be collected and the amount of procollagen peptide secretion quantified using a sandwich enzyme linked immuno-sorbant assay (ELISA) from TAKARA (#MK101).

Elastin Stimulation Assay: Elastin is a connective tissue protein that helps skin resume shape after stretching or contracting. Elastin is also an important load-bearing protein used in places where mechanical energy is required to be stored. Elastin is made by linking many soluble tropoelastin protein molecules, in a reaction catalyzed by lysyl oxidase. Elastin secretion and elastin fibers can be monitored in cultured human fibroblasts by staining of cultured human fibroblasts using immunofluorescent antibodies directed against elastin.

Laminin and Fibronectin Stimulation Assay: Laminin and fibronectin are major proteins in the dermal-epidermal junction (DEJ) (also referred to as the basement membrane). The DEJ is located between the dermis and the epidermis interlocks forming fingerlike projections called rete ridges. The cells of the epidermis receive their nutrients from the blood vessels in the dermis. The rete ridges increase the surface area of the epidermis that is exposed to these blood vessels and the needed nutrients. The DEJ provides adhesion of the two tissue compartments and governs the structural integrity of the skin. Laminin and fibronectin are two structural glycoproteins located in the DEJ. Considered the glue that holds the cells together, laminin and fibronectin are secreted by dermal fibroblasts to help facilitate intra-and inter-cellular adhesion of the epidermal calls to the DEJ. Laminin and fibronectin secretion can be monitored by quantifying laminin and fibronectin in cell supernatants of cultured human fibroblasts treated for 3 days with culture medium with or without 1.0% final concentration of the test ingredient(s). Following incubation, laminin and fibronectin content can be measured using immunofluorescent antibodies directed against laminin and antibodies directed against fibronectin in an enzyme linked immuno-sorbant assay (ELISA). Measurements are normalized for cellular metabolic activity, as determined by bioconversion of 3-(4,5-dimethylthiazol-2-yl)-5-(3-carboxymethoxyphenyl)-2-(4-sulfophenyl)-2H-tetrazolium (MTS).

Tumor Necrosis Factor Alpha (TNF-α) Assay: The prototype ligand of the TNF superfamily, TNF-α, is a pleiotropic cytokine that plays a central role in inflammation. Increase in its expression is associated with an up regulation in pro-inflammatory activity. This bioassay can be used to analyze the effect of any one of the active ingredients, combination of ingredients, or compositions having said combinations disclosed in the specification on the production of TNF-α by human epidermal keratinocytes. The endpoint of this assay can be a spectrophotometric measurement that reflects the presence of TNF-α and cellular viability. The assay employs the quantitative sandwich enzyme immunoassay technique whereby a monoclonal antibody specific for TNF-α has been pre-coated onto a microplate. Standards and samples can be pipetted into the wells and any TNF-α present is bound by the immobilized antibody. After washing away any unbound substances, an enzyme-linked polyclonal antibody specific for TNF-α can be added to the wells. Following a wash to remove any unbound antibody-enzyme reagent, a substrate solution can be added to the wells and color develops in proportion to the amount of TNF-α bound in the initial step using a microplate reader for detection at 450 nm. The color development can be stopped and the intensity of the color can be measured. Subconfluent normal human adult keratinocytes (Cascade Biologics) cultivated in EPILIFE™ standard growth medium (Cascade Biologics) at 37° C. in 5% CO₂, can be treated with phorbol 12-myristate 13-acetate (PMA, 10 ng/ml, SIGMA CHEMICAL, #P1585-1 MG) and any one of the active ingredients, combination of ingredients, or compositions having said combinations disclosed in the specification for 6 hours. PMA has been shown to cause a dramatic increase in TNF-α secretion which peaks at 6 hours after treatment. Following incubation, cell culture medium can be collected and the amount of TNF-a secretion quantified using a sandwich enzyme linked immuno-sorbant assay (ELISA) from R&D Systems (#DTA00C).

Antioxidant (AO) Assay: An in vitro bioassay that measures the total anti-oxidant capacity of any one of the ingredients, combination of ingredients, or compositions having said combinations disclosed in the specification. The assay relies on the ability of antioxidants in the sample to inhibit the oxidation of ABTS® (2,2′-azino-di-[3-ethylbenzthiazoline sulphonate]) to ABTS®·+ by metmyoglobin. The antioxidant system of living organisms includes enzymes such as superoxide dismutase, catalase, and glutathione peroxidase; macromolecules such as albumin, ceruloplasmin, and ferritin; and an array of small molecules, including ascorbic acid, α-tocopherol, β-carotene, reduced glutathione, uric acid, and bilirubin. The sum of endogenous and food-derived antioxidants represents the total antioxidant activity of the extracellular fluid. Cooperation of all the different antioxidants provides greater protection against attack by reactive oxygen or nitrogen radicals, than any single compound alone. Thus, the overall antioxidant capacity may give more relevant biological information compared to that obtained by the measurement of individual components, as it considers the cumulative effect of all antioxidants present in plasma and body fluids. The capacity of the antioxidants in the sample to prevent ABTS® oxidation is compared with that of Trolox, a water-soluble tocopherol analogue, and is quantified as molar Trolox equivalents. Anti-Oxidant capacity kit #709001 from CAYMAN CHEMICAL (Ann Arbor, Michigan USA) can be used as an in vitro bioassay to measure the total anti-oxidant capacity of each of any one of the active ingredients, combination of ingredients, or compositions having said combinations disclosed in the specification. The protocol can be followed according to manufacturer recommendations.

ORAC Assay: Oxygen Radical Absorption (or Absorbance) Capacity (ORAC) of any one of the active ingredients, combination of ingredients, or compositions having said combinations disclosed in the specification can also be assayed by measuring the antioxidant activity of such ingredients or compositions. Antioxidant activity indicates a capability to reduce oxidizing agents (oxidants). This assay quantifies the degree and length of time it takes to inhibit the action of an oxidizing agent, such as oxygen radicals, that are known to cause damage to cells (e.g., skin cells). The ORAC value of any one of the active ingredients, combination of ingredients, or compositions having said combinations disclosed in the specification can be determined by methods known to those of ordinary skill in the art (see U.S. Publication Nos. 2004/0109905 and 2005/0163880; and commercially available kits such as Zen-Bio ORAC Anti-oxidant Assay kit (#AOX-2)). The Zen-Bio ORAC Anti-oxidant Assay kit measures the loss of fluorescein fluorescence over time due to the peroxyl-radical formation by the breakdown of AAPH (2,2′-axobis-2-methyl propanimidamide, dihydrochloride). Trolox, a water soluble vitamin E analog, serves as positive control inhibition fluorescein decay in a dose dependent manner.

Mushroom tyrosinase activity assay: In mammalian cells, tyrosinase catalyzes two steps in the multi-step biosynthesis of melanin pigments from tyrosine (and from the polymerization of dopachrome). Tyrosinase is localized in melanocytes and produces melanin (aromatic quinone compounds) that imparts color to skin, hair, and eyes. Purified mushroom tyrosinase (from SIGMA) can be incubated with its substrate L-Dopa (from FISHER) in the presence or absence of each of the active ingredients, any one of the combination of ingredients, or compositions having said combinations disclosed in the specification. Pigment formation can be evaluated by colorimetric plate reading at 490 nm. The percent inhibition of mushroom tyrosinase activity can be calculated compared to non-treated controls to determine the ability of test ingredients or combinations thereof to inhibit the activity of purified enzyme. Test extract inhibition was compared with that of kojic acid (SIGMA).

Matrix Metalloproteinase 3 and 9 Enzyme Activity (MMP3; MMP9) Assay: An in vitro matrix metalloprotease (MMP) inhibition assay. MMPs are extracellular proteases that play a role in many normal and disease states by virtue of their broad substrate specificity. MMP3 substrates include collagens, fibronectins, and laminin; while MMP9 substrates include collagen VII, fibronectins and laminin. Using Colorimetric Drug Discovery kits from BioMol International for MMP3 (AK-400) and MMP-9 (AK-410), this assay is designed to measure protease activity of MMPs using a thiopeptide as a chromogenic substrate (Ac-PLG-[2-mercapto-4-methyl-pentanoyl]-LG-OC2H5)5,6. The MMP cleavage site peptide bond is replaced by a thioester bond in the thiopeptide. Hydrolysis of this bond by an MMP produces a sulfhydryl group, which reacts with DTNB [5,5′-dithiobis(2-nitrobenzoic acid), Ellman's reagent] to form 2-nitro-5-thiobenzoic acid, which can be detected by its absorbance at 412 nm (ε=13,600 M-1 cm-1 at pH 6.0 and above 7). The active ingredients, any one of the combination of ingredients, or compositions having said combinations disclosed in the specification can be assayed.

Matrix Metalloproteinase 1 Enzyme Activity (MMP1) Assay: An in vitro matrix metalloprotease (MMP) inhibition assay. MMPs are extracellular proteases that play a role in many normal and disease states by virtue of their broad substrate specificity. MMP1 substrates include collagen IV. The MOLECULAR PROBES ENZ/CHEK GELATINASE/COLLAGENASE ASSAY kit (#E12055) utilizes a fluorogenic gelatin substrate to detect MMP1 protease activity. Upon proteolytic cleavage, bright green fluorescence is revealed and may be monitored using a fluorescent microplate reader to measure enzymatic activity.

The ENZ/CHEK GELATINASE/COLLAGENASE ASSAY kit (#E12055) from Invitrogen is designed as an in vitro assay to measure MMP1 enzymatic activity. The active ingredients, any one of the combination of ingredients, or compositions having said combinations disclosed in the specification can be assayed. The assay relies upon the ability of purified MMP1 enzyme to degrade a fluorogenic gelatin substrate. Once the substrate is specifically cleaved by MMP1 bright green fluorescence is revealed and may be monitored using a fluorescent microplate reader. Test materials are incubated in the presence or absence of the purified enzyme and substrate to determine their protease inhibitor capacity.

Cyclooxygenase (COX) Assay: An in vitro cyclooxygenase-1 and -2 (COX-1, -2) inhibition assay. COX is a bifunctional enzyme exhibiting both cyclooxygenase and peroxidase activities. The cyclooxygenase activity converts arachidonic acid to a hydroperoxy endoperoxide (Prostaglandin G2; PGG2) and the peroxidase component reduces the endoperoxide (Prostaglandin H2; PGH2) to the corresponding alcohol, the precursor of prostaglandins, thromboxanes, and prostacyclins. This COX Inhibitor screening assay measures the peroxidase component of cyclooxygenases. The peroxidase activity is assayed colorimetrically by monitoring the appearance of oxidized N,N,N′,N′-tetramethyl-p-phenylenediamine (TMPD). This inhibitor screening assay includes both COX-1 and COX-2 enzymes in order to screen isozyme-specific inhibitors. The Colormetric COX (ovine) Inhibitor screening assay (#760111, CAYMAN CHEMICAL) can be used to analyze the effects of each of the active ingredients, any one of the combination of ingredients, or compositions having said combinations disclosed in the specification on the activity of purified cyclooxygnase enzyme (COX-1 or COX-2). According to manufacturer instructions, purified enzyme, heme and test extracts can be mixed in assay buffer and incubated with shaking for 15 min at room temperature. Following incubation, arachidonic acid and colorimetric substrate can be added to initiate the reaction. Color progression can be evaluated by colorimetric plate reading at 590 nm. The percent inhibition of COX-1 or COX-2 activity can be calculated compared to non-treated controls to determine the ability of test extracts to inhibit the activity of purified enzyme.

Lipoxygenase (LO) Assay: An in vitro lipoxygenase (LO) inhibition assay. LOs are non-heme iron-containing dioxygenases that catalyze the addition of molecular oxygen to fatty acids. Linoleate and arachidonate are the main substrates for LOs in plants and animals. Arachadonic acid may then be converted to hydroxyeicosotrienenoic (HETE) acid derivatives, that are subsequently converted to leukotrienes, potent inflammatory mediators. This assay provides an accurate and convenient method for screening lipoxygenase inhibitors by measuring the hydroperoxides generated from the incubation of a lipoxygenase (5-, 12-, or 15-LO) with arachidonic acid. The Colorimetric LO Inhibitor screening kit (#760700, CAYMAN CHEMICAL) can be used to determine the ability of each of the active ingredients, any one of the combination of ingredients, or compositions having said combinations disclosed in the specification to inhibit enzyme activity. Purified 15-lipoxygenase and test ingredients can be mixed in assay buffer and incubated with shaking for 10 min at room temperature. Following incubation, arachidonic acid can be added to initiate the reaction and the mixtures can be incubated for an additional 10 min at room temperature. Colorimetric substrate can be added to terminate catalysis and color progression can be evaluated by fluorescence plate reading at 490 nm. The percent inhibition of lipoxyganse activity can be calculated compared to non-treated controls to determine the ability of each of the active ingredients, any one of the combination of ingredients, or compositions having said combinations disclosed in the specification to inhibit the activity of purified enzyme.

Elastase Assay: ENZCHEK® Elastase Assay (Kit #E-12056) from MOLECULAR PROBES (Eugene, Oregon USA) can be used as an in vitro enzyme inhibition assay for measuring inhibition of elastase activity for each of the active ingredients, any one of the combination of ingredients, or compositions having said combinations disclosed in the specification. The ENZCHEK kit contains soluble bovine neck ligament elastin that can be labeled with dye such that the conjugate's fluorescence can be quenched. The non-fluorescent substrate can be digested by elastase or other proteases to yield highly fluorescent fragments. The resulting increase in fluorescence can be monitored with a fluorescence microplate reader. Digestion products from the elastin substrate have absorption maxima at ˜505 nm and fluorescence emission maxima at ˜515 nm. The peptide, N-methoxysuccinyl-Ala-Ala-Pro-Val-chloromethyl ketone, can be used as a selective, collective inhibitor of elastase when utilizing the ENZCHEK ELASTASE ASSAY KIT for screening for elastase inhibitors.

Production of Ceramides: Ceramides in cell or tissue samples can be labeled with a mouse monoclonal antibody anti-ceramide (ENZO LIFE SCIENCE, ref ALX-804-196 clone MID15B4) diluted to 1/50 for 2 hours at room temperature with an amplifier system biotin/streptavidin. Video microscope observation can be performed to view ceramides (pink stain).

Oil Control Assay: An assay to measure reduction of sebum secretion from sebaceous glands and/or reduction of sebum production from sebaceous glands can be assayed by using standard techniques known to those having ordinary skill in the art. In some instances, the forehead can be used. Each of the active ingredients, any one of the combination of ingredients, or compositions having said combinations disclosed in the specification can be applied to one portion of the forehead once or twice daily for a set period of days (e.g., 1, 2, 3, 4, 5, 6, 7, 8, 9, 10, 11, 12, 13, 14, or more days), while another portion of the forehead is not treated with the composition. After the set period of days expires, then sebum secretion can be assayed by application of fine blotting paper to the treated and untreated forehead skin. This is done by first removing any sebum from the treated and untreated areas with moist and dry cloths. Blotting paper can then be applied to the treated and untreated areas of the forehead, and an elastic band can be placed around the forehead to gently press the blotting paper onto the skin. After 2 hours the blotting papers can be removed, allowed to dry and then transilluminated. Darker blotting paper correlates with more sebum secretion (or lighter blotting paper correlates with reduced sebum secretion.

Erythema Assay: An assay to measure the reduction of skin redness can be evaluated using a MINOLTA chroma meter. Skin erythema may be induced by applying a 0.2% solution of sodium dodecyl sulfate on the forearm of a subject. The area is protected by an occlusive patch for 24 hrs. After 24 hrs, the patch is removed and the irritation-induced redness can be assessed using the a* values of the MINOLTA chroma meter. The a* value measures changes in skin color in the red region. Immediately after reading, the area is treated with the active ingredients, any one of the combination of ingredients, or compositions having said combinations disclosed in the specification. Repeat measurements can be taken at regular intervals to determine the formula's ability to reduce redness and irritation.

Skin Moisture/Hydration Assay: Skin moisture/hydration benefits can be measured by using impedance measurements with the Nova Dermal Phase Meter. The impedance meter measures changes in skin moisture content. The outer layer of the skin has distinct electrical properties. When skin is dry it conducts electricity very poorly. As it becomes more hydrated increasing conductivity results. Consequently, changes in skin impedance (related to conductivity) can be used to assess changes in skin hydration. The unit can be calibrated according to instrument instructions for each testing day. A notation of temperature and relative humidity can also be made. Subjects can be evaluated as follows: prior to measurement they can equilibrate in a room with defined humidity (e.g., 30-50%) and temperature (e.g., 68-72° C.). Three separate impedance readings can be taken on each side of the face, recorded, and averaged. The T5 setting can be used on the impedance meter which averages the impedance values of every five seconds application to the face. Changes can be reported with statistical variance and significance. Each of the active ingredients, any one of the combination of ingredients, or compositions having said combinations disclosed in the specification can be assayed according to this process.

Skin Clarity and Reduction in Freckles and Age Spots Assay: Skin clarity and the reduction in freckles and age spots can be evaluated using a Minolta Chromometer. Changes in skin color can be assessed to determine irritation potential due to product treatment using the a* values of the Minolta Chroma Meter. The a* value measures changes in skin color in the red region. This is used to determine whether each of the active ingredients, any one of the combination of ingredients, or compositions having said combinations disclosed in the specification is inducing irritation. The measurements can be made on each side of the face and averaged, as left and right facial values. Skin clarity can also be measured using the Minolta Meter. The measurement is a combination of the a*, b, and L values of the Minolta Meter and is related to skin brightness, and correlates well with skin smoothness and hydration. Skin reading is taken as above. In one non-limiting aspect, skin clarity can be described as L/C where C is chroma and is defined as (a²+b²)^{1/2}.

Skin Dryness, Surface Fine Lines, Skin Smoothness, and Skin Tone Assay: Skin dryness, surface fine lines, skin smoothness, and skin tone can be evaluated with clinical grading techniques. For example, clinical grading of skin dryness can be determined by a five point standard Kligman Scale: (0) skin is soft and moist; (1) skin appears normal with no visible dryness; (2) skin feels slightly dry to the touch with no visible flaking; (3) skin feels dry, tough, and has a whitish appearance with some scaling; and (4) skin feels very dry, rough, and has a whitish appearance with scaling. Evaluations can be made independently by two clinicians and averaged.

Clinical Grading of Skin Tone Assay: Clinical grading of skin tone can be performed via a ten point analog numerical scale: (10) even skin of uniform, pinkish brown color. No dark, erythremic, or scaly patches upon examination with a hand held magnifying lens. Microtexture of the skin very uniform upon touch; (7) even skin tone observed without magnification. No scaly areas, but slight discolorations either due to pigmentation or erythema. No discolorations more than 1 cm in diameter; (4) both skin discoloration and uneven texture easily noticeable. Slight scaliness. Skin rough to the touch in some areas; and (1) uneven skin coloration and texture. Numerous areas of scaliness and discoloration, either hypopigmented, erythremic or dark spots. Large areas of uneven color more than 1 cm in diameter. Evaluations were made independently by two clinicians and averaged.

Clinical Grading of Skin Smoothness Assay: Clinical grading of skin smoothness can be analyzed via a ten point analog numerical scale: (10) smooth, skin is moist and glistening, no resistance upon dragging finger across surface; (7) somewhat smooth, slight resistance; (4) rough, visibly altered, friction upon rubbing; and (1) rough, flaky, uneven surface. Evaluations were made independently by two clinicians and averaged.

Skin Smoothness and Wrinkle Reduction Assay With Methods Disclosed in Packman et al. (1978): Skin smoothness and wrinkle reduction can also be assessed visually by using the methods disclosed in Packman et al. (1978). For example, at each subject visit, the depth, shallowness and the total number of superficial facial lines (SFLs) of each subject can be carefully scored and recorded. A numerical score was obtained by multiplying a number factor times a depth/width/length factor. Scores are obtained for the eye area and mouth area (left and right sides) and added together as the total wrinkle score.

Skin Firmness Assay with a Hargens Ballistometer: Skin firmness can be measured using a Hargens ballistometer, a device that evaluates the elasticity and firmness of the skin by dropping a small body onto the skin and recording its first two rebound peaks. The ballistometry is a small lightweight probe with a relatively blunt tip (4 square mm-contact area) was used. The probe penetrates slightly into the skin and results in measurements that are dependent upon the properties of the outer layers of the skin, including the stratum corneum and outer epidermis and some of the dermal layers.

Skin Softness/Suppleness Assay with a Gas Bearing Electrodynamometer: Skin softness/suppleness can be evaluated using the Gas Bearing Electrodynamometer, an instrument that measures the stress/strain properties of the skin. The viscoelastic properties of skin correlate with skin moisturization. Measurements can be obtained on the predetermined site on the cheek area by attaching the probe to the skin surface with double-stick tape. A force of approximately 3.5 gm can be applied parallel to the skin surface and the skin displacement is accurately measured. Skin suppleness can then be calculated and is expressed as DSR (Dynamic Spring Rate in gm/mm).

Appearance of Lines and Wrinkles Assay with Replicas: The appearance of lines and wrinkles on the skin can be evaluated using replicas, which is the impression of the skin's surface. Silicone rubber like material can be used. The replica can be analyzed by image analysis. Changes in the visibility of lines and wrinkles can be objectively quantified via the taking of silicon replicas form the subjects' face and analyzing the replicas image using a computer image analysis system. Replicas can be taken from the eye area and the neck area, and photographed with a digital camera using a low angle incidence lighting. The digital images can be analyzed with an image processing program and are of the replicas covered by wrinkles or fine lines was determined.

Surface Contour of the Skin Assay with a Profilometer/Stylus Method: The surface contour of the skin can be measured by using the Profilometer/Stylus method. This includes either shining a light or dragging a stylus across the replica surface. The vertical displacement of the stylus can be fed into a computer via a distance transducer, and after scanning a fixed length of replica a cross-sectional analysis of skin profile can be generated as a two-dimensional curve. This scan can be repeated any number of times along a fix axis to generate a simulated 3-D picture of the skin. Ten random sections of the replicas using the stylus technique can be obtained and combined to generate average values. The values of interest include Ra which is the arithmetic mean of all roughness (height) values computed by integrating the profile height relative to the mean profile height. Rt which is the maximum vertical distance between the highest peak and lowest trough, and Rz which is the mean peak amplitude minus the mean peak height. Values are given as a calibrated value in mm. Equipment should be standardized prior to each use by scanning metal standards of know values. Ra Value can be computed by the following equation: Rₐ=Standardize roughness; lₘ =the traverse (scan) length; and y=the absolute value of the location of the profile relative to the mean profile height (x-axis).

MELANODERM™ Assay: In other non-limiting aspects, the efficacy of each of the active ingredients, any one of the combination of ingredients, or compositions having said combinations disclosed in the specification can be evaluated by using a skin analog, such as, for example, MELANODERM™. Melanocytes, one of the cells in the skin analog, stain positively when exposed to L-dihydroxyphenyl alanine (L-DOPA), a precursor of melanin. The skin analog, MELANODERM™, can be treated with a variety of bases containing each of the active ingredients, any one of the combination of ingredients, or compositions having said combinations disclosed in the specification or with the base alone as a control. Alternatively, an untreated sample of the skin analog can be used as a control.

Production of Filaggrin: Changes in the production of filaggrin in keratinocytes due to each of the active ingredients, any one of the combination of ingredients, or compositions having said combinations disclosed in the specification can be measured. Filaggrin is the precursor to Natural Moisturizing Factor (NMF) in the skin. Increased NMF increases the moisture content of the skin. Filaggrin production in treated and non-treated keratinocytes can be determined using a bioassay that analyzes filaggrin concentration in keratinocyte cell lysates. A non-limiting example of a bioassay that can be used to quantify filaggrin production is the PROTEINSIMPLE® SIMON™ western blotting protocol. For each sample, normal human epidermal keratinocytes (NHEK) are grown in EPI-200—MATTEK EPILIFE® growth media with calcium from Life Technologies (M-EP-500-CA). NHEK are incubated in growth medium overnight at 37° C. in 5% CO₂ prior to treatment. NHEK are then incubated in growth medium with 1% test compound/extract or no compound/extract (negative control) for 24 to 36 hours. The NHEK can then be washed, collected, and stored on ice or colder until lysed on ice using a lysis buffer and sonication. The protein concentrations of the samples can be determined and used to normalize the samples. The lysates can be stored at −80° C. until use in the quantification assay.

The PROTEINSIMPLE® SIMON™ western blotting bioassay assay employs a quantitative western blotting immunoassay technique using an antibody specific for filaggrin to quantitatively detect filaggrin in the test samples. Cell samples are lysed and normalized for protein concentration. Normalized samples and molecular weight standards can then be loaded and ran on a denatured protein separation gel using capillary electrophoresis. The proteins in the gel are immobilized and immunoprobed using a primary antibody specific for filaggrin. The immobilized proteins can then be immunoprobed with an enzyme-linked detection antibody that binds the primary antibody. A chemiluminescent substrate solution can then be added to the immobilized proteins to allow chemiluminescent development in proportion to the amount of filaggrin bound in the immobilization. The chemiluminescent development is stopped at a specific time and the intensity of the chemiluminescent signal can be measured and compared to positive and negative controls.

Production of Occludin: Changes in the production of occludin in keratinocytes due to each of the active ingredients, any one of the combination of ingredients, or compositions having said combinations disclosed in the specification can be measured. Occludin is a protein critical to the formulation of tight junctions and the skin's moisture barrier function. A non-limiting example of how occludin production in treated and non-treated keratinocytes can be determined is by the use of a bioassay that analyzes occludin concentration in keratinocyte cell lysates. The bioassay can be performed using PROTEINSIMPLE® SIMON™ western blotting protocol. For the samples, adult human epidermal keratinocytes (HEKa) from Life Technologies (C-005-5C) can be grown at 37° C. and 5% CO2 for 24 hours in EPILIFE™ growth media with calcium from Life Technologies (M-EP-500-CA) supplemented with Keratinocyte Growth Supplement (HKGS) from Life Technologies (S-101-5). HEKa are then incubated in growth medium with test compound/extract, no compound/extract for negative control, or with 1 mM CaCl₂ for positive control for 24 to 48 hours. The HEKa are then washed, collected, and stored on ice or colder until lysed on ice using a lysis buffer and sonication. The protein concentrations of the samples can be determined and used to normalize the samples. The lysates are stored at −80° C. until use in the bioassay.

The PROTEINSIMPLE® SIMON™ western blotting bioassay assay employs a quantitative western blotting immunoassay technique using an antibody specific for occludin to quantitatively detect occludin in the test samples. Cell samples are lysed and normalized for protein concentration. Normalized samples and molecular weight standards are then loaded and ran on a denatured protein separation gel using capillary electrophoresis. The proteins in the gel are then immobilized and immunoprobed using a primary antibody specific for occludin. The immobilized proteins are immunoprobed with an enzyme-linked detection antibody that binds the primary antibody. A chemiluminescent substrate solution is then added to the immobilized proteins to allow chemiluminescent development in proportion to the amount of occludin bound in the immobilization. The chemiluminescent development can be stopped at a specific time and the intensity of the chemiluminescent signal can be measured and compared to positive and negative controls.

Keratinocyte Monolayer Permeability: Changes in the permeability of a keratinocyte monolayer due to each of the active ingredients, any one of the combination of ingredients, or compositions having said combinations disclosed in the specification can be measured. Keratinocyte monolayer permeability is a measure of skin barrier integrity. Keratinocyte monolayer permeability in treated and non-treated keratinocytes can be determined using, as a non-limiting example, the In Vitro Vascular Permeability assay by MILLIPORE (ECM642). This assay analyzes endothelial cell adsorption, transport, and permeability. Briefly, adult human epidermal keratinocytes from Life Technologies (C-005-5C) can be seeded onto a porous collagen-coated membrane within a collection well. The keratinocytes are then incubated for 24 hours at 37° C. and 5% CO₂ in EPILIFE™ growth media with calcium from LIFE TECHNOLOGIES (M-EP-500-CA) supplemented with Keratinocyte Growth Supplement (HKGS) from LIFE TECHNOLOGIES (S-101-5). This incubation time allows the cells to form a monolayer and occlude the membrane pores. The media is then replaced with fresh media with (test sample) or without (non-treated control) test compounds/extracts and the keratinocytes are incubated for an additional 48 hours at 37° C. and 5% CO₂. To determine permeability of the keratinocyte monolayer after incubation with/without the test compound/extract, the media is replaced with fresh media containing a high molecular weight Fluorescein isothiocyanate (FITC)-Dextran and the keratinocytes are incubated for 4 hours at 37° C. and 5% CO₂. During the 4 hours incubation, FITC can pass through the keratinocytes monolayer and porous membrane into the collection well at a rate proportional to the monolayer's permeability. After the 4 hour incubation, cell viability and the content of FITC in the collection wells can be determined. For the FITC content, the media in the collection well is collected and fluorescence of the media determined at 480 nm (Em) when excited at 520 nm. Percent permeability and percent change in comparison to the non-treated controls can be determined by the following equations: Percent Permeability=((Mean Ex/Em of test sample)/Mean Ex/Em untreated control)*100; Percent Change=Percent Permeability of test sample−Percent Permeability of untreated control.

Production of Hyaluronic Acid: Changes in the production of hyaluronic acid in human dermal fibroblasts due to each of the active ingredients, any one of the combination of ingredients, or compositions having said combinations disclosed in the specification can be measured. HA is a polysaccharide involved in stabilization of the structure of the matrix and is involved in providing turgor pressure to tissue and cells.

As one non-limiting example, HA production in treated and non-treated adult human dermal fibroblasts (HDFa) cells can be determined using the Hyaluronan DuoSet ELISA kit from R&D Systems (DY3614). In this assay, for production of samples, subconfluent HDFa cells from Cascade Biologics (C-13-5C) are incubated at 37° C. and 10% CO₂ in starvation medium (0.15% fetal bovine serum and 1% Penicillin Streptomycin solution in Dulbecco's Modified Eagle Medium) for 72 hours prior to treatment. The cells are then incubated with fresh starvation medium with either test compound, positive control (phorbol 12-myristate 13-acetate from SIGMA-ALDRICH (P1585) and platelet derived growth factor from SIGMA-ALDRICH (P3201)), or no additive for 24 hours. Media is then collected and frozen at −80° C. until use in the ELISA assay.

Briefly, the ELISA assay employs a quantitative sandwich enzyme immunoassay technique whereby a capture antibody specific for HA can be pre-coated onto a microplate. Standards and media from treated and untreated cells are pipetted into the microplate wells to enable any HA present to be bound by the immobilized antibody. After washing away any unbound substances, an enzyme-linked detection antibody specific for HA is added to the wells. Following a wash to remove any unbound antibody-enzyme reagent, a substrate solution is added to the wells to allow color development in proportion to the amount of HA bound in the initial step. The color development is stopped at a specific time and the intensity of the color at 450 nm can be measured using a microplate reader.

As another non-limiting example, human skin explants can be cultured in survival explants medium at 37° C. in a humidified atmosphere supplemented with 5% CO2. Treatment of the explants can be carried out by topical application of sample product (n=3) on days D0, D2, D3, D6, D8, and D9. The control explants (n=3) receive no treatment except renewal of survival explants medium. Half of the volume of the survival medium can be renewed at days D3, D6, and D8. At D9, three explants of each condition can be taken and cut in half. A half explant is fixed in buffered formalin and the other is frozen at −80° C.

After 48 hours of fixation in ordinary Bouin and 24 hours in formalin, the samples can be dried and soaked in paraffin using an automatic tissue processor Leica TP 1020. Sections of 5 microns can be performed with a microtome (Minot type LEICA RM2125) and mounted on SUPERFROST™ histological slides. Microscopic observations can be performed by optical microscopy, using a LEICA ORTHOPLAN or LEICA DM LB microscope. Images can be taken with an OLYMPUS DP72 camera and CELL\hat{ }D software. General morphology can be examined on paraffin sections stained with Masson's trichrome Goldner variant. The staining of hyaluronic acid can be performed with an anti-hyaluronic acid biotinylated protein (HABP) (SEIKAGAKU ref 400763-1A) diluted to 1/100 for 1 hour at room temperature, with an amplifier system biotin/streptavidin (VECTOR, VECTASTAIN PK-7200).

Inhibition of Hyaluronidase Activity: Changes in the activity of hyaluronidase due to each of the active ingredients, any one of the combination of ingredients, or compositions having said combinations disclosed in the specification can be measured. Hyaluronidase is an enzyme that degrades HA. HA is a polysaccharide involved in stabilization of the structure of the matrix and is involved in providing turgor pressure to tissue and cells. As one non-limiting example, hyaluronidase activity can be determined using an in vitro protocol modified from SIGMA-ALDRICH protocol #EC 3.2.1.35. Briefly, hyaluronidase type 1-S from SIGMA-ALDRICH (H3506) is added to microplate reaction wells containing test compound or controls. Tannic acid can be used as a positive control inhibitor, no test compound can be added for the control enzyme, and wells with test compound or positive control but without hyaluronidase can be used as a background negative control. The wells are incubated at 37° C. for 10 minutes before addition of substrate (HA). Substrate is added and the reactions incubated at 37° C. for 45 minutes. A portion of each reaction solution is then transferred to and gently mixed in a solution of sodium acetate and acetic acid pH 3.75 to stop that portion of the reaction (stopped wells). The stopped wells and the reaction wells should both contain the same volume of solution after addition of the portion of the reaction solution to the stopped wells. Both the reaction wells and the stopped wells are incubated for 10 minutes at room temperature. Absorbance at 600 nm is then measured for both the reaction wells and the stopped wells. Inhibition can be calculated using the following formulas: Inhibitor (or control) activity=(Inhibitor stopped wells absorbance at 600 nm−inhibitor reaction wells absorbance at 600 nm); Initial activity=control enzyme absorbance at 600 nm;

$$Percent Inhibition = [ ( Initial activity / Inhibitor Activity ) * 100 ] - 10 \; 0 .$$

Peroxisome Proliferator-Activated Receptor Gamma (PPAR-γ) Activity: Changes in the activity of PPAR-γ due to each of the active ingredients, any one of the combination of ingredients, or compositions having said combinations disclosed in the specification can be measured. PPAR-γ is a receptor critical for the production of sebum. As one non-limiting example, the activity of PPAR-γ can be determined using a bioassay that analyzes the ability of a test compound or composition to inhibit binding of a ligand. Briefly, fluorescent small-molecule pan-PPAR ligand, FLUORMONE™ Pan-PPAR Green, available from Life Technologies (PV4894), can be used to determine if test compounds or compositions are able to inhibit binding of the ligand to PPAR-γ. The samples wells include PPAR-γ and fluorescent ligand and either: test compound or composition (test); a reference inhibitor, rosiglitazone (positive control); or no test compound (negative control). The wells are incubated for a set period of time to allow the ligand opportunity to bind the PPAR-γ. The fluorescence polarization of each sample well can then be measured and compared to the negative control well to determine the percentage of inhibition by the test compound or composition.

Cytokine array: Human epidermal keratinocytes are cultured to 70-80% confluency. The media in the plate is aspirated and 0.025% trypsin/EDTA is added. When the cells became rounded, the culture dish is gently tapped to release the cells. The trypsin/EDTA containing cells are removed from the culture dish and neutralized. Cells are centrifuged for 5 min. at 180×g to form a pellet of cells. The supernatant is aspirated. The resulting pellet is resuspended in EPILIFE™ media (Cascade Biologics). The cells are seeded in 6-well plates at approximately 10-20% confluency. After the cells became approximately 80% confluent, the media is aspirated and 1.0 ml of EPILIFE™, along with phorbol 13-Myristate 12-acetate (“PMA”) (a known inducer of inflammation) and the test composition dilutions are added to two replicate wells (i.e., 1.0% (100 μl of 100X stock) and 0.1% (10 μl of 100X stock) test compositions are diluted into a final volume of 1 ml EPILIFE™ Growth Medium). The media is gently swirled to ensure adequate mixing. In addition, 1.0 ml of EPILIFE™ is added to the control wells, with and without additional PMA. The plates are then incubated at 37±1° C. and 5.0±1% CO₂ for approximately 5 hours after dosing. Following this 5-hour incubation, all media is collected in conical tubes and frozen at −70° C.

For analysis, a 16-pad hybridization chamber is attached to 16-pad FAST slides arrayed in triplicate with 16 anti-cytokine antibodies plus experimental controls (WHATMAN BIOSCIENCES), and the slides are placed into a FASTFrame (4 slides per frame) for processing. Arrays are blocked for 15 min. at room temperature using 70 ml S&S PROTEIN ARRAY BLOCKING BUFFER (WHATMAN SCHLEICHER AND SCHEULL). Blocking buffer is removed and 70 ml of each supernatant sample is added to each array. Arrays are incubated for 3 hours at room temperature with gentle agitation. Arrays are washed 3 times with TBS-T. Arrays are treated with 70 ml of an antibody cocktail, containing one biotinylated antibody corresponding to each of the arrayed capture antibodies. Arrays are incubated for 1 hour at room temperature with gentle agitation. Arrays are washed 3 times with TBS-T. Arrays are incubated with 70 ml of a solution containing streptavidin-Cy5 conjugate for 1 hour at room temperature with gentle agitation. Arrays are washed 3 times with TBS-T, quickly rinsed in de-ionized water, and dried.

Slides can be imaged in a PERKIN-ELMER SCANARRAY 4000 confocal fluorescent imaging system. Array images can be saved and analyzed using IMAGING RESEARCH ARRAYVISION software. Briefly, spot intensities are determined by subtracting background signal. Spot replicates from each sample condition can be averaged and then compared to the appropriate controls.

Endothelial Tube Formation: Endothelial tube formation is involved in angiogenesis and micro-vessel capillary formation. Capillary formation and angiogenesis may contribute to redness and rosacea of the skin. The ability for endothelial cells to form tubes in the presence or absence of test extracts and compounds may be determined using a capillary tubule disruption assay with pre-formed primary human umbilical vein endothelial cells (HUVEC) in a cell culture system.

Briefly, HUVECs are cultured in vitro on Extracellular Matrix, which stimulates the attachment and tubular morphogenesis of endothelial cells to form capillary-like lumen structures. These in vitro formed capillary tubules are similar to human blood vessel capillaries in many aspects. The capillary tube assay is based on this phenomenon and is used for evaluation of potential vasculature targeting agents.

HUVEC cultures are grown in a 5% CO₂ 37° C. cell incubator. The full growth medium for HUVECs is Endothelial Cell Basal Medium (EBM) supplemented with 2% fetal bovine serum (FBS), 12 μg/ml bovine brain extract, 1 μg/ml hydrocortisone, and 1 μg/ml GA-1000 (gentamicin-amphothericin). HUVEC cultures between passage 3 and 8 may be used for all assay experiments.

HUVECs are pre-labeled with fluorescent agent Calcein AM and seeded in Extracellular Matrix coated 96-well culture plate with their full growth medium. After about four hours of the morphogenesis process, the endothelial capillary tubes should be formed. Then, test agent in designed doses in 50 μl volume is applied into the formed capillary tubule cultures as treatment conditions. The no-treatment controls can be added with vehicle of test agents. SUTENT®, a FDA approved anti-angiogenic drug one concentration can be included as assay performance control. After about six hours of treatment, the endothelial tubule morphology in each well is examined by microscopy, imaged, and the capillary disrupting activities under treatment conditions can be quantitatively analyzed. Each test conditions can be conducted in duplicate wells, including controls.

### Example 3

### Consumer Testing for Luminous Foundation

### Background

The inventors formulated a luminous foundation described herein by removing/changing several raw materials from previously developed foundations and replacing a preservative system described herein due to D5/D6 regulations. Additional changes include removing powders which contain microplastics. The wider project goals are to improve coverage & wear, minimize dry down time, and minimize shade change from wet to dry.

### Objective

(1) Determine which claims can be validated during a consumer home usage test, and (2) obtain voluntary testimonials for those that meet criteria.

### Methodology

A total of 1× masked/unbranded test product was included in this study. Respondents were instructed to replace their current liquid luminous foundation with the test product while maintaining their skin care routine. Respondents completed the following surveys: Day 1 AM Survey—Beginning of Day 1, application & appearance for immediate claims. Day 7 AM Survey—Beginning of Day 7, overall assessment of application & appearance claims. Day 7 PM Survey—End of Day 7, appearance, wear & removal of foundation claims.

### Screening Criteria

Total N=196 Healthy females. Current user (4× a week) of luminous liquid foundation, light to medium and medium to full coverage. Must be a skin shade acceptor. Skin Tone Quotas: Light (n=50), Medium (n=50) and Deep (n=50). 75% of sample must have a skin concern: fine lines or wrinkles [extreme or severe not eligible for the study]; dark spots/blemishes; uneven skin tone; and/or visible pores.

Mix of: Ages 25-55; Ethnicity quotas Latina women (n=50), African-American (n=50) Self-assessed skin type: dry skin (75%) and oily combo skin (25%). Not pregnant, nursing, or planning to become pregnant. No known skin conditions, sensitivities, and/or infections. Not currently using any physician-prescribed products on the face Has not participated and is not currently participating in a cosmetic, skincare or clinical study involving product applied to the face for the past 3 months. Does not work for a cosmetic, market research or consumer testing company. Must be willing to use products over the required 7 days and complete 3 total surveys. Able to read, write, and understand local language; sign a consent form.

### Summary

Initial Use (Day 1) consumers found the foundation: instantly provides weightless coverage; instantly provides good coverage; instantly hydrates their skin; instantly provides a seamless match to their skin tone; instantly enhances their complexion; instantly creates a smooth finish; instantly evens out skin tone; instantly creates a natural looking finish; instantly creates a glowing finish; instantly provides a natural radiance; instantly provides a luminous finish; skin instantly appears brighter; skin instantly looks smoother; instantly reduces the appearance of blemishes; instantly reduces the appearance of dark spots; instantly smooths the appearance of fine lines; instantly blurs the look of pores; instantly reduces the appearance of redness.

After one week of use consumers, in the morning, found the foundation: applies smoothly; blends evenly; easy to spread; applies easily; dries quickly; provides weightless coverage; provides good coverage; provides buildable coverage; provides medium to full coverage; achieved the desired look wanted with ease; provides a seamless match to their skin tone; enhances their complexion; helped achieve a flawless look; creates a smooth finish; evens out skin tone; creates a natural looking finish; creates a glowing finish; provides a natural radiance; skin appears brighter; skin looks healthier; skin looks smoother; reduces the appearance of blemishes; does not crease in expression lines; reduces the appearance of dark spots; provides a luminous finish; smooths the appearance of fine lines; smooths the appearance of wrinkles; blurs the look of pores; reduces the appearance of redness; feels comfortable on skin; and feels lightweight.

Additionally, after one week of use consumers, in the evening, found the foundation: suitable for their skin type; skin looks radiant all day; skin feels healthy; skin looks natural; does not feel drying; face looks polished; provides long lasting hydration; does not crease in expression lines; provides long lasting coverage; provides long lasting luminous finish; looks fresh all day; wears all day; does not flake; does not cake; does not fade; transfer resistant; removes easily; removes quickly; removes effortlessly without tugging; able to fully remove the liquid foundation.

### Example 4

### Additional Testing for Luminous Foundation

### Objective

Determine if formulation improvements for coverage with the light shade is consistent across medium and dark shades when compared to both the lab control and production control.

### Methodology

Six (6) trained descriptive sensory panelists sequentially, monadically evaluated three (3) replications of each product of the four (4) test products. Samples were pipetted to the panelists for evaluation using an Eppendorf in set quantities. Application: Visual 200 microliters, Pick-Up 400 microliters, Application 50 microliters The ballot had 39 attributes. A 0-100 intensity scale was used to evaluate the attributes (where 0 means “none” and 100 means “extreme”) except for number of rubs which is a count (1-12+), and dry time which is counted in seconds after application: 2 visual; 2 pick-up; 7 application; 14 immediate post application; 13 10-minute post application (add 10 min shine); 1 Product Transfer

Evaluations were conducted at The Mary Kay Lewisville office using the Skin feel trained panel. Data was analyzed using SIMS/R Tukey's ANOVA mean separation at p<0.05 for statistical significance.

### Summary

### General

Medium & Dark Shade sensory testing among the TW3D Luminous Foundation revealed some statistical differences among the prototypes when compared to both controls. However, most of these differences were small and not consumer noticeable. While there were consumer noticeable differences found among Visually Thick and Ease of Pick-Up when comparing the Beige Lab and Production Medium shade controls, Ease of Pick-Up was the only consumer noticeable difference found among the Dark shade Bronze Lab and Production controls.

Among the Medium shade prototypes, consumer noticeable differences were found for Visually Thick, Ease of Pick-Up, Immediate Coverage, Thickness, and Spreadability.

Dark shade prototypes sensory results indicate consumer noticeable differences among Visually Thick, Ease of Pick-Up, Denseness (during rub-out), and Thickness.

### Coverage

Coverage among the Medium shade was statistically improved for both prototypes immediately after application (but not 10 minutes post application. Improvements to Immediate Coverage was only a consumer noticeable difference when comparing the Lead prototype (AB7524) to the Beige Lab Control. Dark shade prototypes had statistical improvements to Coverage at both timepoints, however, these differences would not be considered consumer noticeable.

### Implications

Unlike the Light Shade sensory findings indicating both statistical and consumer noticeable differences made to Coverage when compared to the control (see EXP 23-AA8970), sensory findings for both Medium and Dark shades show mostly statistical improvements made to Coverage when compared to the control.

It is important to note that the Coverage for Light Shade controls was between the mid-40 range (especially by 10 minutes post application), whereas Coverage for Medium and Dark shade controls was at/around 50-range on a 0-100 intensity scale.

When taking into account all sensory testing across the multiple shades, Coverage appears higher across the shades; new prototypes are at the mid-50 to 60 range across Light, Medium, and Dark shades as opposed to the controls were between the mid-40 to 50 range for Coverage.

### Example 5

### Sensory Testing for Luminous Foundation

### Objective

Determine (1) which prototype increases coverage, (2) which prototype has reduced dry down time, and (3) investigate differences among lab batch control and production control.

### Methodology

Six (6) trained descriptive sensory panelists sequentially, monadically evaluated three (3) replications of each product of the six (6) test products. Samples were pipetted to the panelists for evaluation using an Eppendorf in set quantities. Application: Visual 200 microliters, Pick-Up 400 microliters, Application 50 microliters The ballot had 38 attributes. A 0-100 intensity scale was used to evaluate the attributes (where 0 means “none” and 100 means “extreme”) except for number of rubs which is a count (1-12+), and dry time which is counted in seconds after application: 2 visual; 2 pick-up; 7 application; 14 immediate post application; 12 10-minute post application; 1 Product Transfer

Evaluations were conducted at The Mary Kay Lewisville office using the Skin feel trained panel. Data was analyzed using SIMS/R Dunnett's ANOVA mean separation at p<0.05 for statistical significance.

### Summary & Implications

### General

Round 5 of TW3D Luminous Foundation sensory testing revealed several statistical differences among the prototypes when compared to both controls. However, most of these differences were small and not consumer noticeable. Among test prototypes, consumer noticeable differences were found among Coverage (at both time points) and Thickness (during application). There were no statistical and no consumer noticeable differences found when the Production Control and Lab Control were compared to each other.

### Coverage

Coverage (immediately and post 10 minutes application) was statistically increased for both AB7515 and AB7518, and these differences would be considered consumer noticeable. AB5713 only had statistical and consumer noticeable improvements to Coverage at 10 minutes and not immediate upon application.

### Dry-Time

Dry-Time was similar across all prototypes indicating no improvements for this attribute.

### Implications

While dry time was not improved among any test protypes, both AB7515 and AB7518 are the BEST prototypes since Coverage was statistically improved [and consumer noticeable] at both timepoints [immediate and 10 minutes post application]. AB5713 is a good prototype since Coverage was statistically improved at 10 minutes post application [consumer noticeable difference]. AB7512 is the LEAST IDEAL prototype as it is the most similar of all prototypes tested to both controls and had no improvements to Coverage.

### Example 6

### Sensory Testing for Matte Foundation

### Objective

Determine (1) which prototype increases coverage, (2) which prototype has reduced dry down time, and (3) investigate differences among lab batch control and production control.

### Methodology

Seven (7) trained descriptive sensory panelists sequentially, monadically evaluated three (3) replications of each product of the six (6) test products. Samples were pipetted to the panelists for evaluation using an Eppendorf in set quantities. Application: Visual 200 microliters, Pick-Up 400 microliters, Application 50 microliters The ballot had 38 attributes. A 0-100 intensity scale was used to evaluate the attributes (where 0 means “none” and 100 means “extreme”) except for number of rubs which is a count (1-12+), and dry time which is counted in seconds after application: 2 visual; 2 pick-up; 7 application; 14 immediate post application; 12 10-minute post application; 1 Product Transfer

Evaluations were conducted at The Mary Kay Lewisville office using the Skin feel trained panel. Data was analyzed using SIMS/R Dunnett's ANOVA mean separation at p<0.05 for statistical significance.

### Summary & Implications

### General

Round 5 of TW3D Matte Foundation sensory testing revealed several statistical differences among the prototypes when compared to both controls. However, most of these differences were small and not consumer noticeable. Among test prototypes, consumer noticeable differences were found only among Visually Thick and Ease of Pick-up. There was one statistical difference found between the Production Control and Lab Control; While the Production Control was statistically visually thicker than the Lab Control, this was a small difference and was not consumer noticeable.

### Coverage

Coverage (both time points) was only statistically improved with test prototypes. However, the differences were small and would not be considered consumer noticeable. Consistent with previous sensory results conducted with the PEC (Round IV), Coverage for the lab control has been within a similar magnitude (e.g., medium-upper 40's).

### Dry-Time

Dry-Time was similar across all prototypes.

### Example 7

### Opacity Testing

The opacity of various foundations, including foundations described herein, are presented in Table 5. A comparison of the measured opacity initially and after 10 minutes is shown.

| | Opacity | |
|---|---|---|
| Trial Name/Product Name | (CR) | Δ% |
| LOREAL TRUE MATCH CLASSIC IVORY N2 | 81.76 | 0.32 |
| LOREAL TRUE MATCH CLASSIC IVORY | 82.08 | |
| N2 10 MIN | | |
| MK CC CREAM MED TO DARK | 95.98 | −0.89 |
| MK CC CREAM MED TO DARK 10 MIN | 95.09 | |
| LOREAL INFALLABLE PRO MATTE 101 | 94.19 | −6.12 |
| CLASSIC IVORY | | |
| LOREAL INFALLABLE PRO MATTE 101 | 88.07 | |
| CLASSIC IVORY 10 MIN | | |
| MAYBELLINE DEWY + SMOOTH 118 | 80.08 | −8.04 |
| LIGHT BEIGE | | |
| MAYBELLINE DEWY + SMOOTH 118 | 72.04 | |
| LIGHT BEIGE 10 MIN | | |
| ALMAY TLC 140 BUFF | 98.02 | −0.31 |
| ALMAY TLC 140 BUFF 10 MIN | 97.71 | |
| MAYBELLINE MATTE + PORELESS | 81.59 | −7.98 |
| 120 CLASSIC IVORY | | |
| MAYBELLINE MATTE + PORELESS | 73.61 | |
| 120 CLASSIC IVORY 10 MIN | | |
| NYX BARE WITH ME CONCEALER | 96.56 | −0.84 |
| SERUM BWMCCS03 VANILLA | | |
| NYX BARE WITH ME CONCEALER | 95.72 | |
| SERUM BWMCCS03 VANILLA 10 MIN | | |
| MK TW 3D MATTE FNDN BEIGE N150 | 94.27 | −10.38 |
| MK TW 3D MATTE FNDN BEIGE N150 10 MIN | 83.89 | |
| DERMABLEND MED NATURAL 40N | 99.05 | −0.60 |
| DERMABLEND MED NATURAL 40N 10 MIN | 98.45 | |
| MK TW 3D MATTE FNDN IVORY C100 | 89.48 | −8.03 |
| MK TW 3D MATTE FNDN IVORY C100 10 MIN | 81.45 | |
| MK TW 3D MATTE FNDN IVORY C110 | 92.52 | −4.79 |
| MK TW 3D MATTE FNDN IVORY C110 10 MIN | 87.73 | |
| MK TW 3D MATTE FNDN IVORY N140 | 95.98 | −2.48 |
| MK TW 3D MATTE FNDN IVORY N140 10 MIN | 93.50 | |
| MK TW 3D MATTE FNDN IVORY N160 | 95.19 | −9.74 |
| MK TW 3D MATTE FNDN IVORY N160 10 MIN | 85.45 | |
| MK TW 3D MATTE FNDN BEIGE C220 | 96.47 | −4.40 |
| MK TW 3D MATTE FNDN BEIGE C220 10 MIN | 92.07 | |
| MK TW 3D MATTE FNDN BEIGE W180 | 93.76 | −2.79 |
| MK TW 3D MATTE FNDN BEIGE W180 10 MIN | 90.97 | |
| MK TW 3D MATTE FNDN BRONZE C180 | 99.77 | −2.97 |
| MK TW 3D MATTE FNDN BRONZE C180 10 MIN | 96.80 | |
| MK TW 3D MATTE FNDN BRONZE C170 | 99.70 | −8.45 |
| MK TW 3D MATTE FNDN BRONZE C170 10 MIN | 91.25 | |
| MK TW 3D MATTE FNDN BRONZE W100 | 98.05 | −11.92 |
| MK TW 3D MATTE FNDN BRONZE W100 10 MIN | 86.13 | |
| MK TW 3D LUM FNDN IVORY C100 | 97.77 | 0.42 |
| MK TW 3D LUM FNDN IVORY C100 10 MIN | 98.19 | |
| MAYBELLINE FIT ME 375 JAVA | 96.13 | — |
| MAYBELLINE FIT ME 375 JAVA 10 MIN | — | |
| MK AT PLAY LIGHT TAN | 83.62 | −12.14 |
| MK AT PLAY LIGHT TAN 10 MIN | 71.48 | |
| MK AT PLAY MEDIUM TO DEEP | 88.01 | −11.24 |
| MK AT PLAY MEDIUM TO DEEP 10 MIN | 76.77 | |
| MK AT PLAY LIGHT TO MEDIUM | 84.74 | −11.65 |
| MK AT PLAY LIGHT TO MEDIUM 10 MIN | 73.09 | |
| MK AT PLAY MEDIUM | 84.66 | −11.43 |
| MK AT PLAY MEDIUM 10 MIN | 73.23 | |
| MK AT PLAY LIGHT | 78.55 | −6.83 |
| MK AT PLAY LIGHT 10 MIN | 71.72 | |
| MK AT PLAY DARK TAN | 93.14 | −5.50 |
| MK AT PLAY DARK TAN 10 MIN | 87.64 | |
| MK AT PLAY VERY LIGHT | 79.62 | −10.81 |
| MK AT PLAY VERY LIGHT 10 MIN | 68.81 | |
| MK AT PLAY SOFT MEDIUM | 82.28 | −15.79 |
| MK AT PLAY SOFT MEDIUM 10 MIN | 66.49 | |
| MK AT PLAY DEEP TAN | 93.01 | −8.10 |
| MK AT PLAY DEEP TAN 10 MIN | 84.91 | |
| MK AT PLAY DEEP | 89.87 | −5.44 |
| MK AT PLAY DEEP 10 MIN | 84.43 | |
| MK FLAWLESS FACE TW 3D LUM FNDN | 99.50 | 0.03 |
| IVORY C100 AB7512 | | |
| MK FLAWLESS FACE TW 3D LUM FNDN | 99.53 | |
| IVORY C100 AB7512 10 MIN | | |
| MK FLAWLESS FACE TW 3D LUM FNDN | 99.45 | −0.68 |
| IVORY C100 AB7513 | | |
| MK FLAWLESS FACE TW 3D LUM FNDN | 98.77 | |
| IVORY C100 AB7513 10 MIN | | |
| MK FLAWLESS FACE TW 3D LUM FNDN | 100.00 | 0.00 |
| IVORY C100 AB7514 | | |
| MK FLAWLESS FACE TW 3D LUM FNDN | 100.00 | |
| IVORY C100 AB7514 10 MIN | | |
| MK FLAWLESS FACE TW 3D LUM FNDN | 99.88 | −0.10 |
| IVORY C100 AB7515 | | |
| MK FLAWLESS FACE TW 3D LUM FNDN | 99.78 | |
| IVORY C100 AB7515 10 MIN | | |
| MK FLAWLESS FACE TW 3D LUM FNDN | 99.73 | 0.27 |
| IVORY C100 AB7518 | | |
| MK FLAWLESS FACE TW 3D LUM FNDN | 100.00 | |
| IVORY C100 AB7518 10 MIN | | |
| MK FLAWLESS FACE TW 3D MATTE | 97.41 | −4.99 |
| FNDN IVORY C100 AB5189 | | |
| MK FLAWLESS FACE TW 3D MATTE | 92.42 | |
| FNDN IVORY C100 AB5189 10 MIN | | |
| MK FLAWLESS FACE TW 3D MATTE | 99.72 | 0.24 |
| FNDN IVORY C100 AB5198 | | |
| MK FLAWLESS FACE TW 3D MATTE | 99.96 | |
| FNDN IVORY C100 AB5198 10 MIN | | |
| MK FLAWLESS FACE TW 3D MATTE | 99.18 | −0.46 |
| FNDN IVORY C100 AB5199 | | |
| MK FLAWLESS FACE TW 3D MATTE | 98.72 | |
| FNDN IVORY C100 AB5199 10 MIN | | |
| MK FLAWLESS FACE TW 3D MATTE | 98.44 | −1.44 |
| FNDN IVORY C100 AB8401 | | |
| MK FLAWLESS FACE TW 3D MATTE | 97.00 | |
| FNDN IVORY C100 AB8401 10 MIN | | |
| MK TW 3D LUM FNDN IVORY C100 | 97.77 | 0.42 |
| MK TW 3D LUM FNDN IVORY C100 10 MIN | 98.19 | |
| MK FLAWLESS FACE TW 3D LUM | 99.50 | 0.03 |
| FNDN IVORY C100 AB7512 | | |
| MK FLAWLESS FACE TW 3D LUM | 99.53 | |
| FNDN IVORY C100 AB7512 10 MIN | | |
| MK FLAWLESS FACE TW 3D LUM | 99.45 | −0.68 |
| FNDN IVORY C100 AB7513 | | |
| MK FLAWLESS FACE TW 3D LUM | 98.77 | |
| FNDN IVORY C100 AB7513 10 MIN | | |
| MK FLAWLESS FACE TW 3D LUM | 100.00 | 0.00 |
| FNDN IVORY C100 AB7514 | | |
| MK FLAWLESS FACE TW 3D LUM | 100.00 | |
| FNDN IVORY C100 AB7514 10 MIN | | |
| MK FLAWLESS FACE TW 3D LUM | 99.88 | −0.10 |
| FNDN IVORY C100 AB7515 | | |
| MK FLAWLESS FACE TW 3D LUM | 99.78 | |
| FNDN IVORY C100 AB7515 10 MIN | | |
| MK FLAWLESS FACE TW 3D LUM | 99.73 | 0.27 |
| FNDN IVORY C100 AB7518 | | |
| MK FLAWLESS FACE TW 3D LUM | 100.00 | |
| FNDN IVORY C100 AB7518 10 MIN | | |
| MK TW 3D MATTE FNDN IVORY C100 | 89.48 | −8.03 |
| MK TW 3D MATTE FNDN IVORY C100 10 MIN | 81.45 | |
| MK FLAWLESS FACE TW 3D MATTE | 97.41 | −4.99 |
| FNDN IVORY C100 AB5189 | | |
| MK FLAWLESS FACE TW 3D MATTE | 92.42 | |
| FNDN IVORY C100 AB5189 10 MIN | | |
| MK FLAWLESS FACE TW 3D MATTE | 99.72 | 0.24 |
| FNDN IVORY C100 AB5198 | | |
| MK FLAWLESS FACE TW 3D MATTE | 99.96 | |
| FNDN IVORY C100 AB5198 10 MIN | | |
| MK FLAWLESS FACE TW 3D MATTE | 99.18 | −0.46 |
| FNDN IVORY C100 AB5199 | | |
| MK FLAWLESS FACE TW 3D MATTE | 98.72 | |
| FNDN IVORY C100 AB5199 10 MIN | | |
| MK FLAWLESS FACE TW 3D MATTE | 98.44 | −1.44 |
| FNDN IVORY C100 AB8401 | | |
| MK FLAWLESS FACE TW 3D MATTE | 97.00 | |
| FNDN IVORY C100 AB8401 10 MIN | | |

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1. A topical skin composition comprising a dimethicone, at least one film-forming agent, and at least one surface-treated pigment, wherein the film-forming agent comprises a dimethicone acrylate copolymer, a trimethicone, and/or isobutylmethacrylate/bis-hydroxypropyl dimethicone copolymer.

2. The topical skin composition of claim 1, wherein the dimethicone comprises a low-molecular weight dimethicone.

3. The topical skin composition of claim 1, wherein the film-forming agent comprises a dimethicone acrylate copolymer and a trimethicone.

4. The topical skin composition of claim 3, wherein the trimethicone comprises methyl trimethicone.

5. The topical skin composition of claim 1, wherein the film-forming agent comprises an isobutylmethacrylate/bis-hydroxypropyl dimethicone copolymer.

6. The topical skin composition of claim 1, wherein the film-forming agent comprises a dimethicone acrylate copolymer dissolved in methyl trimethicone.

7. The topical skin composition of claim 1, wherein the film-forming agent comprises a isobutylmethacrylate/bis-hydroxypropyl dimethicone copolymer dissolved in isoparaffin.

8. The topical skin composition of claim 1, wherein the surface-treated pigments comprise a metal oxide.

9. The topical skin composition of claim 1, wherein the surface-treated pigments are in a dimethicone dispersion.

10. The topical skin composition of claim 1, wherein the dimethicone is between 0.65 to 10 centistokes.

11. The topical skin composition of claim 1, wherein the composition further comprises a soft-focus agent.

12. The topical skin composition of claim 1, wherein the composition further comprises a mattifying agent.

13. The topical skin composition of claim 1, wherein the composition is an emulsion.

14. The topical skin composition of claim 1, wherein the composition does not comprise decamethylcyclopentasiloxane.

15. The topical skin composition of claim 1, wherein the composition does not comprise a resin.

16. A method of treating skin, the method comprising applying the topical skin composition of claim 1 topically to skin of an individual.

17. The method of claim 16, wherein the applying results in camouflaging of dyschromia and/or skin imperfections on the individual.

18. The method of claim 16, wherein the applying results in a luminous effect on the skin of the individual.

19. The method of claim 16, wherein the applying results in a mattifying effect on the skin of the individual.

20. The method of claim 16, wherein the skin of the individual is oily.

21. The method of claim 16, wherein the topical skin composition absorbs sebum on the skin of the individual.
